(19) United States
(12) Patent Application Publication
(10) Pub. No.: US 2009/0275594 A1
(43) Pub. Date: Nov. 5, 2009

(54) 3-HYDRAZONE PIPERAZINYL RIFAMYCIN DERIVATIVES USEFUL AS ANTIMICROBIAL AGENTS

(76) Inventors: Mark J. MACIELAG, Branchburg, NJ (US); Manomi Tennakoon, Hillsborough, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/434,832

(22) Filed: May 4, 2009

Related U.S. Application Data
(60) Provisional application No. 61/050,330, filed on May 5, 2008.

(51) Int. Cl. A61K 31/496; C07D 413/14; A61P 31/04

(52) U.S. Cl. 514/253.1; 544/367; 514/254.04; 544/364

(57) ABSTRACT

The present invention is directed to novel 3-hydrazone piperazinyl rifamycin derivatives, pharmaceutical compositions containing them and the use of said derivatives and pharmaceutical compositions as antimicrobial agents against pathogenic microorganisms, particularly against resistant microbes.

### CROSS REFERENCE TO RELATED APPLICATIONS

This application claims priority of the benefits of the filing of U.S. Provisional Application Ser. No. 61/050,330, filed May 5, 2008. The complete disclosures of the aforementioned related U.S. patent application is/are hereby incorporated herein by reference for all purposes.

### FIELD OF THE INVENTION

The present invention is directed to novel 3-hydrazone piperazinyl rifamycin derivatives, pharmaceutical compositions containing them and the use of said derivatives and pharmaceutical compositions as antimicrobial agents against pathogenic microorganisms, particularly against resistant microbes.

### BACKGROUND OF THE INVENTION

The chemical and medical literature describes compounds that are said to be antimicrobial, i.e., capable of destroying or suppressing the growth or reproduction of microorganisms, such as bacteria. For example, such antibacterial agents are described in Antibiotics, Chemotherapeutics, and Antibacterial Agents for Disease Control (M. Greyson, editor, 1982), E. Gale et al., The Molecular Basis of Antibiotic Action 2d edition (1981), Recent Research Developments in Antimicrobial Agents & Chemotherapy (S. G. Pandalai, Editor, 2001), Quinolone Antimicrobial Agents (John S Wolfson, David C Hooper, Editors, 1989), and F. O'Grady, H. P. Lambert, R. G. Finch, D. Greenwood, Martin Dedicoat, “Antibiotic and Chemotherapy, 7th edn.” (1997).

The mechanisms of action of these antibacterial agents vary. However, they are generally believed to function in one or more ways: by inhibiting cell wall synthesis or repair; by altering cell wall permeability; by inhibiting protein synthesis; or by inhibiting the synthesis of nucleic acids. For example, beta-lactam antibacterial agents act through inhibiting essential penicillin binding proteins (PBPs) in bacteria, which are responsible for cell wall synthesis. As another example, quinolones act, at least in part by inhibiting synthesis of DNA, thus preventing the cell from replicating.

The pharmacological characteristics of antimicrobial agents, and their suitability for any given clinical use, vary. For example, the classes of antimicrobial agents (and members within a class) may vary in 1) their relative efficacy against different types of microorganisms, 2) their susceptibility to development of microbial resistance and 3) their pharmacological characteristics such as their bioavailability and biodistribution. Accordingly, selection of an appropriate antimicrobial agent in a given clinical situation requires analysis of many factors, including the type of organism involved, the desired method of administration, the location of the infection to be treated and other considerations.

However, many such attempts to produce improved antimicrobial agents yield equivocal results. Indeed, few antimicrobial agents are produced that are truly clinically acceptable in terms of their spectrum of antimicrobial activity, avoidance of microbial resistance, and pharmacology. Thus there is a continuing need for broad-spectrum antimicrobial agents, which are effective against resistant microbes.

Examples of bacterial infections resistant to antibiotic therapy have been reported in the past; they are now a significant threat to public health in the developed world. The development of microbial resistance (perhaps as a result of the intense use of antibacterial agents over extended periods of time) is of increasing concern in medical science. “Resistance” can be defined as existence of organisms, within a population of a given microbial species, that are less susceptible to the action of a given antimicrobial agent. This resistance is of particular concern in environments such as hospitals and nursing homes, where relatively high rates of infection and intense use of antibacterial agents are common. See, e.g., W. Sanders, Jr. et al., “Inducible Beta-lactamases: Clinical and Epidemiologic Implications for the Use of Newer Cephalosporins”, Review of Infectious Diseases, p. 830 (1988).

Pathogenic bacteria are known to acquire resistance via several distinct mechanisms including inactivation of the antibiotic by bacterial enzymes (e.g., β-lactamases hydrolyzing penicillin and cephalosporins); removal of the antibiotic using efflux pumps; modification of the target of the antibiotic via mutation and genetic recombination (e.g., penicillin-resistance in Neiserria gonorrhoeae); and acquisition of a readily transferable gene from an external source to create a resistant target (e.g., methicillin-resistance in Staphylococcus aureus). There are certain Gram-positive pathogens, such as vancomycin-resistant Enterococcus faecium, which are resistant to most commercially available antibiotics.

Hence existing antibacterial agents have limited capacity in overcoming the threat of resistance. Thus it would be advantageous to provide new antibacterial agents that can be used against resistant microbes.

### SUMMARY OF THE INVENTION

The present invention is directed to compounds of formula (I)

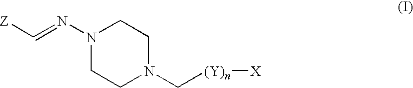

wherein

Z is selected from the group consisting of

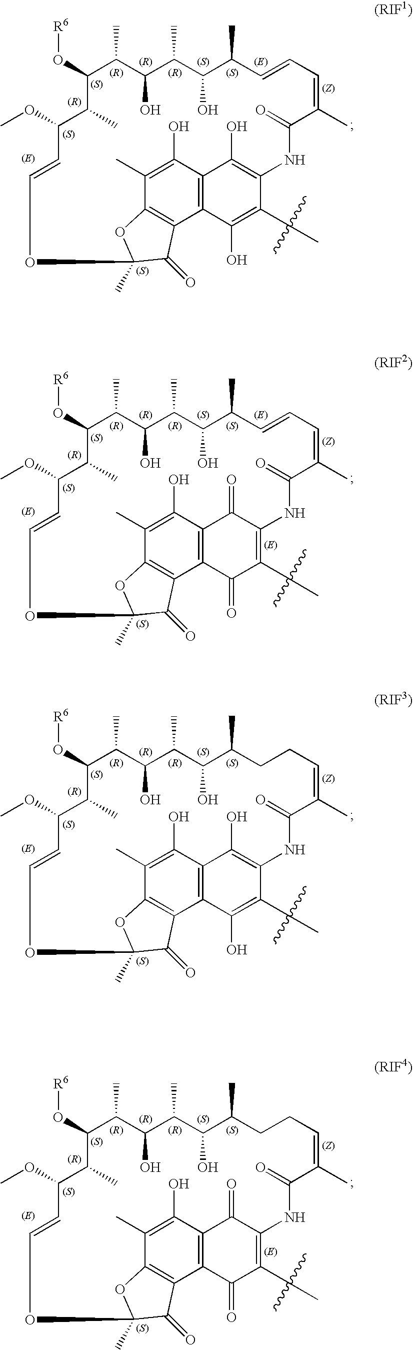

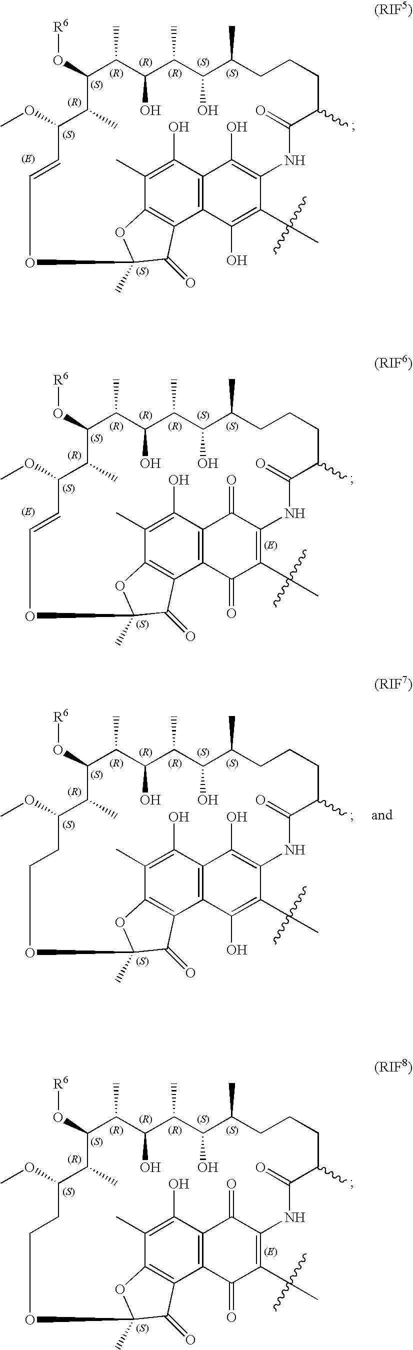

R⁶ is selected from the group consisting of hydrogen and acyl;

n is an integer from 0 to 1;

Y is selected from the group consisting of C₁₋₄alkyl and C₂₋₆alkenyl;

X is selected from the group consisting of

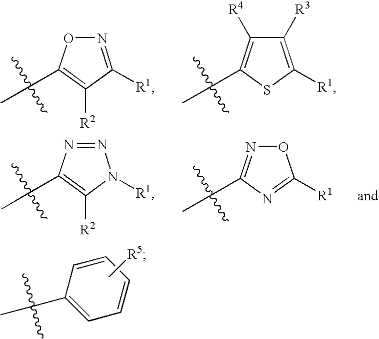

R¹ is selected from the group consisting of C₁₋₄alkyl, —C₁₋₄alkyl-OH, —C₁₋₄alkyl-NRARB, aryl, heteroaryl, —CO₂H and —CO₂—C₁₋₄alkyl;

wherein the aryl is optionally substituted with one to two substituents independently selected from the group consisting of halogen, C₁₋₄alkyl, C₁₋₄alkoxy, halogenated C₁₋₄alkyl, halogenated C₁₋₄alkoxy, —NR^{C}—C(O)—C₁₋₄alkyl and —O—C₁₋₄alkyl-NR^{C}R^{D};

wherein R^{A}, R^{B}, R^{C} and R^{D} are each independently selected from the group consisting of hydrogen and C₁₋₄alkyl;

R² is selected from the group consisting of hydrogen, —CO₂H and —CO₂—C₁₋₄alkyl;

R³ and R⁴ are each independently selected from the group consisting of hydrogen, halogen, C₁₋₄alkyl, aryl, heteroaryl, —CO₂H and —CO₂—C₁₋₄alkyl;

R⁵ is selected from the group consisting of hydrogen, halogen, C₁₋₄alkyl, aryl, heteroaryl, —CO₂H and —CO₂—C₁₋₄alkyl;

and pharmaceutically acceptable salts, esters and prodrugs thereof.

Illustrative of the invention is a pharmaceutical composition comprising a pharmaceutically acceptable carrier and the product prepared according to the process described herein. An illustration of the invention is a pharmaceutical composition made by mixing the product prepared according to the process described herein and a pharmaceutically acceptable carrier. Illustrating the invention is a process for making a pharmaceutical composition comprising mixing the product prepared according to the process described herein and a pharmaceutically acceptable carrier.

It has been found that the compounds of this invention, and compositions containing these compounds, are effective antimicrobial agents against a broad range of pathogenic microorganisms with advantages of activity against resistant microbes.

Accordingly, the present invention is also directed to a method of treating a subject having a condition caused by or contributed to by bacterial infection, which comprises administering to said mammal a therapeutically effective amount of the compound of Formula (I).

The present invention is further directed to a method of preventing a subject from suffering from a condition caused by or contributed to by bacterial infection, which comprises administering to the subject a prophylactically effective dose of the pharmaceutical composition of a compound of Formula (I).

The present invention is further directed to the use of a compound of formula (I) for the preparation of a medicament for treating and/or preventing a condition caused by or contributed to by bacteria infection, in a subject in need thereof. In an embodiment, the present invention is directed to the use of a compound of formula (I) for the preparation of a medicament for treating and/or preventing a condition caused by or contributed to by bacteria infection associated with a drug resistant bacteria, in a subject in need thereof.

### DETAILED DESCRIPTION OF THE INVENTION

The present invention is directed to a process for the preparation of compound of formula (I)

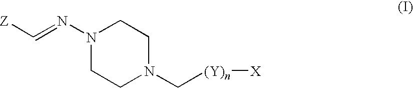

wherein Z, n, Y and X are as herein defined; and pharmaceutically acceptable salts, esters and prodrugs thereof. The compounds of formula (I) are useful as antimicrobial agents against pathogenic microorganisms, preferably, resistant microbes.

In an embodiment of the present invention, Z is selected from the group consisting of (RIF¹) and (RIF²); preferably Z is (RIF¹).

In an embodiment of the present invention R⁶ is hydrogen. In another embodiment of the present invention, R⁶ is acyl, preferably, R⁶ is selected from the group consisting of —C(O)—(C₁₋₄alkyl). In another embodiment of the present invention, R⁶ is —C(O)—CH₃.

In an embodiment of the present invention, n is 0. In another embodiment of the present invention, n is 1.

In an embodiment of the present invention, Y is selected from the group consisting of C₁₋₄alkyl and C₂₋₄alkenyl. In another embodiment of the present invention, Y is selected from the group consisting of C₁₋₄alkyl. In another embodiment of the present invention, Y is selected from the group consisting of —CH₂—, —CH₂CH₂— and —CH₂CH₂CH₂—.

In an embodiment of the present invention, X is selected from the group consisting of

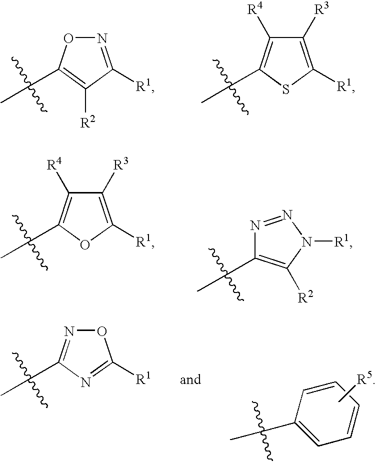

In another embodiment of the present invention, X is

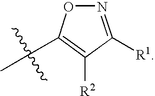

In an embodiment of the present invention, R¹ is selected from the group consisting of C₁₋₄alkyl, —C₁₋₄alkyl-OH, —C₁₋₄alkyl-NR^{A}R^{B}, aryl, heteroaryl, —CO₂H and —CO₂—C₁₋₄alkyl; wherein the aryl is optionally substituted with one or more substituents independently selected from the group consisting of halogen, C₁₋₄alkyl, C₁₋₄alkoxy, halogenated C₁₋₄alkyl, halogenated C₁₋₄alkoxy, —NR^{C}—C(O)—C₁₋₄alkyl and —O—C₁₋₄alkyl-NR^{C}R^{D}; and wherein R^{A}, R^{B}, R^{C} and R^{D} are each independently selected from the group consisting of hydrogen and C₁₋₂alkyl.

In another embodiment of the present invention, R¹ is selected from the group consisting of C₁₋₄alkyl, —C₁₋₄alkyl-OH, —C₁₋₄alkyl-NR^{A}R^{B}, aryl and heteroaryl; wherein the aryl is optionally substituted with one to two substituents independently selected from the group consisting of halogen, C₁₋₄alkyl, fluorinated C₁₋₄alkyl, fluorinated C₁₋₄alkoxy, —NR^{C}—C(O)—C₁₋₄alkyl and —O—C₁₋₄alkyl-NR^{C}R^{D} ; and wherein R^{A}, R^{B}, R^{C} and R^{D} are each independently selected from the group consisting of hydrogen and C₁₋₂alkyl.

In another embodiment of the present invention, R¹ is selected from the group consisting of methyl, hydroxymethyl, dimethylaminomethyl-, phenyl, 4-chlorophenyl, 3-chlorophenyl, 2-chloro[phenyl, 2,4-dichlorophenyl, 4-fluorophenyl, 3,4-difluorophenyl, 4-methylphenyl, 4-(trifluoromethyl)-phenyl, 4-(trifluoromethoxy)-phenyl, 4-(methylcarbonylamino)-phenyl, 4-(dimethylaminopropoxy)-phenyl, 2-pyridyl, 3-pyridyl and 4-pyridyl.

In another embodiment of the present invention, R¹ is selected from the group consisting of methyl, hydroxymethyl, dimethylamino-methyl-, phenyl, 4-fluorophenyl, 4-(trifluoromethyl)-phenyl, 4-(trifluoromethoxy)-phenyl, 4-(dimethylaminopropoxy)-phenyl, 4-(methylcarbonylamino)-phenyl, 2-pyridyl, 3-pyridyl and 4-pyridyl. In another embodiment of the present invention, R¹ is selected from the group consisting of methyl, hydroxymethyl, dimethylaminomethyl-, 2-pyridyl, 3-pyridyl, 4-pyridyl and 4-(methylcarbonylamino)-phenyl.

In an embodiment of the present invention, R² is selected from the group consisting of hydrogen, —CO₂H and —CO₂—C₁₋₄alkyl. In another embodiment of the present invention, R² is selected from the group consisting of hydrogen, —CO₂H and —CO₂—C₁₋₂alkyl. In another embodiment of the present invention, R² is selected from the group consisting of hydrogen, —CO₂H and —CO₂—CH₃. In another embodiment of the present invention, R² is hydrogen.

In an embodiment of the present invention, R³ and R⁴ are each independently selected from the group consisting of hydrogen, C₁₋₄alkyl, phenyl, monocyclic heteroaryl, —CO₂H and —CO₂—C₁₋₄alkyl. In another embodiment of the present invention, R³ and R⁴ are each hydrogen.

In an embodiment of the present invention, R⁵ is selected from the group consisting of hydrogen, halogen, C₁₋₄alkyl, phenyl, monocyclic heteroaryl, —CO₂H and —CO₂—C₁₋₄alkyl. In another embodiment of the present invention, R⁵ is selected from the group consisting of halogen. In another embodiment of the present invention, R⁵ is chloro.

In another embodiment of the present invention, X is selected from the group consisting of 5-(3-phenyl-isoxazolyl), 5-(3-(4-chlorophenyl)-isoxazolyl), 5-(3-(3-chlorophenyl)-isoxazolyl), 5-(3-(2,4-dichlorophenyl)-isoxazolyl), 5-(3-(4-fluorophenyl)-isoxazolyl), 5-(3-(3,4-difluorophenyl)-isoxazolyl), 5-(3-(4-trifluoromethylphenyl)-isoxazolyl), 5-(3-(4-methylphenyl)-isoxazolyl), 5-(3-(4-methoxyphenyl)-isoxazolyl), 5-(3-methyl-isoxazlyl), 5-(3-(hydroxymethyl-isoxazolyl), 5-(3-dimethylaminomethyl-isoxazolyl), 5-(3-(2-pyridyl)-isoxazolyl), 5-(3-(3-pyridyl)-isoxazolyl), 5-(3-(4-pyridyl)-isoxazolyl), 5-(3-(4-methylcarbonyl-amino-phenyl)-isoxazolyl), 5-{3-[4-(3-dimethylaminopropoxy)-phenyl]-isoxazolyl}, 5-(3-(4-chlorophenyl)-4-methoxycarbonyl-isoxazolyl), 5-(3-(4-chlorophenyl)-4-carboxy-isoxazolyl), 2-(5-phenyl-thineyl), 2-(5-(4-chlorophenyl)-isoxazolyl), 2-(5-(2-chlorophenyl)-furyl), 2-(5-(4-chlorophenyl)-furyl), 4-(1-(4-chlorophenyl)-[1,2,3]triazolyl), 3-(5-(4-chlorophenyl)-[1,2,4]oxadiazolyl) and 4-chlorophenyl.

Additional embodiments of the present invention, include those wherein the substituents selected for one or more of the variables defined herein (i.e. Z, n, Y and X) are independently selected to be any individual substituent or any subset of substituents selected from the complete list as defined herein.

Representative compounds of the present invention are as listed in Table 1, below. In another embodiment of the present invention is any single compound or subset of compounds selected from the representative compounds listed in Table 1.

TABLE 1
Representative Compounds of Formula (I)

| ID No. | Structure | R⁶ |
|---|---|---|
| 1 | 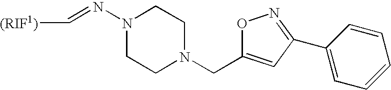 | —C(O)—CH₃ |
| 2 | 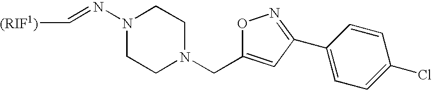 | —C(O)—CH₃ |
| 3 | 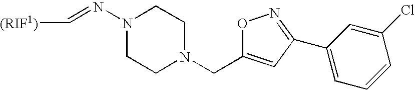 | —C(O)—CH₃ |
| 4 | 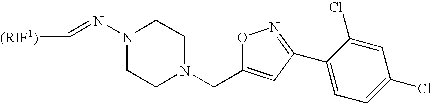 | —C(O)—CH₃ |
| 5 | 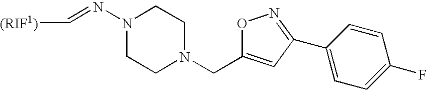 | —C(O)—CH₃ |
| 6 | 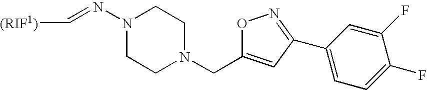 | —C(O)—CH₃ |
| 7 | 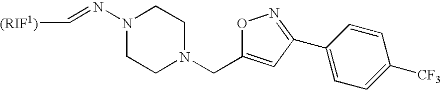 | —C(O)—CH₃ |
| 8 | 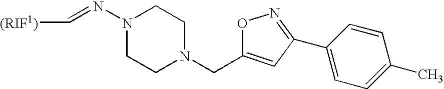 | —C(O)—CH₃ |
| 9 | 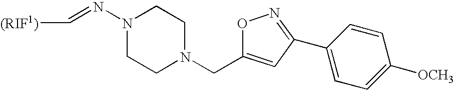 | —C(O)—CH₃ |
| 10 | 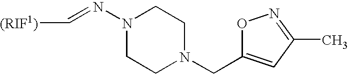 | —C(O)—CH₃ |
| 11 | 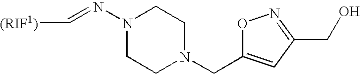 | —C(O)—CH₃ |
| 12 | 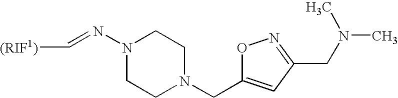 | —C(O)—CH₃ |
| 13 | 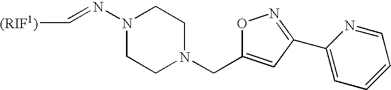 | —C(O)—CH₃ |
| 14 | 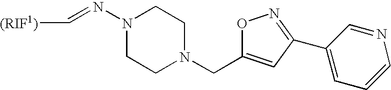 | —C(O)—CH₃ |
| 15 | 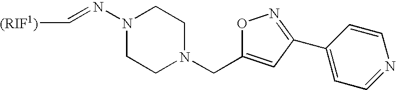 | —C(O)—CH₃ |
| 16 | 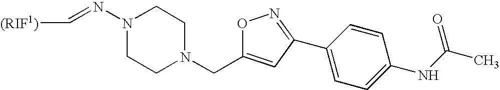 | —C(O)—CH₃ |
| 17 | 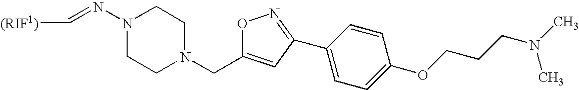 | —C(O)—CH₃ |
| 18 | 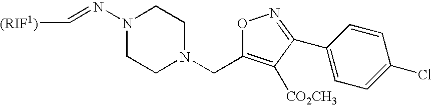 | —C(O)—CH₃ |
| 19 | 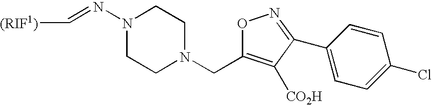 | —C(O)—CH₃ |
| 20 | 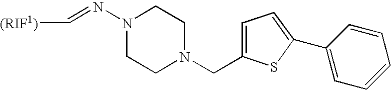 | —C(O)—CH₃ |
| 21 | 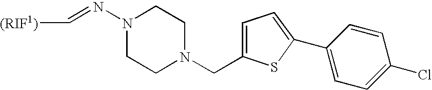 | —C(O)—CH₃ |
| 22 | 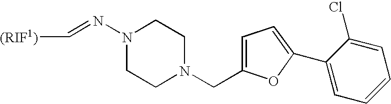 | —C(O)—CH₃ |
| 23 | 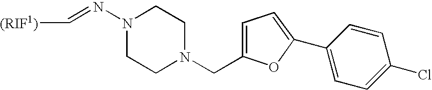 | —C(O)—CH₃ |
| 24 | 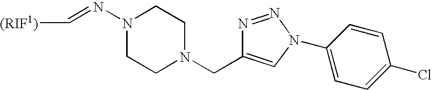 | —C(O)—CH₃ |
| 25 | 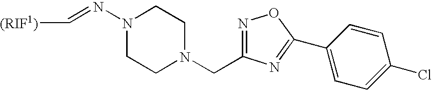 | —C(O)—CH₃ |
| 26 | 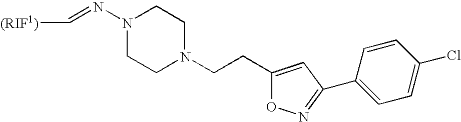 | —C(O)—CH₃ |
| 27 | 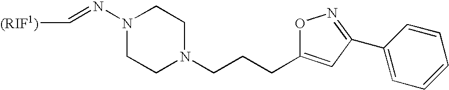 | —C(O)—CH₃ |
| 28 | 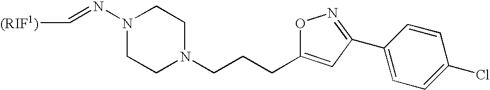 | —C(O)—CH₃ |
| 29 | 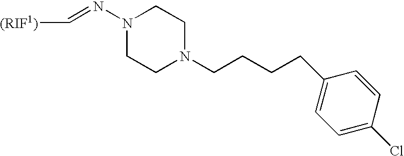 | —C(O)—CH₃ |
| 30 | 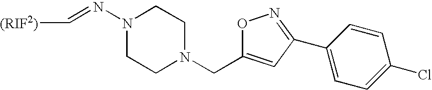 | —C(O)—CH₃ |
| 31 | 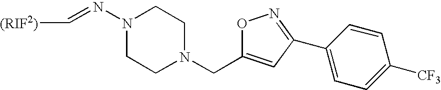 | —C(O)—CH₃ |

In an embodiment, the present invention is directed to compounds of formula (I) whose MIC (minimum inhibitory concentration) against strain A as measured according to the procedure described in Example 32 is less than or equal to about 4 μg/mL, preferably less than or equal to about 0.5 μg/mL, more preferably less than or equal to about 0.06 μg/mL. In an embodiment, the present invention is directed to compounds of formula (I) whose MIC (minimum inhibitory concentration) against strain B as measured according to the procedure described in Example 32 is less than or equal to about 2 μg/mL, preferably less than or equal to about 0.25 μg/mL, more preferably less than or equal to about 0.03 μg/mL. In an embodiment, the present invention is directed to compounds of formula (I) whose MIC (minimum inhibitory concentration) against strain C as measured according to the procedure described in Example 32 is less than or equal to about 2 μg/mL, preferably less than or equal to about 0.25 μg/mL, more preferably less than or equal to about 0.03 μg/mL. In an embodiment, the present invention is directed to compounds of formula (I) whose MIC (minimum inhibitory concentration) against strain D as measured according to the procedure described in Example 32 is less than or equal to about 4 μg/mL, preferably less than or equal to about 0.5 μg/mL, more preferably less than or equal to about 0.03 μg/mL.

As used herein, the terms “halo” or “halogen” shall mean fluoro, chloro, bromo or iodo; preferably fluoro, chloro or bromo.

As used herein, the term “alkyl” shall mean a saturated, straight or branched hydrocarbon chain having 1 to 15 carbons. For example, alkyl radicals include methyl, ethyl, propyl, isopropyl, butyl, isobutyl, sec-butyl, t-butyl, pentyl and the like. The term “halogenated alkyl” shall mean any alkyl group as defined above substituted with one to five halogen atoms, preferably with at least one halogen atom, preferably substituted with a least one fluoro atom. Suitable examples include but are not limited to —CF₃, —CH₂—CF₃, —CF₂—CF₂—CF₂—CF₃, and the like. Similarly, the term “fluorinated alkyl” shall mean any alkyl group as defined above substituted with one to five fluoro atoms, preferably with at least one fluoro atom. Suitable examples include but are not limited to —CF₃, —CH₂—CF₃, —CF₂—CF₂—CF₂—CF₃, and the like.

The term “alkenyl” shall mean a straight or branched hydrocarbon chain having at least one carbon-carbon double bond and having 2 to 15 carbon atoms.

As used herein, the prefix “Cₓ-C_{y}” wherein x and y are numbers shall denote the number of carbon atoms present in a particular functional group. For example, the term “C₁-C₄alkyl” denotes any straight or branched chain alkyl as herein defined of between 1 and 4 carbon atoms, inclusive. Similarly, the term “C₂-C₄alkenyl” shall denote an alkenyl group of between 2 and 4 carbon atoms inclusive.

The term “alkoxy” shall denote an oxygen ether radical of the above described straight or branched chain alkyl groups (i.e. a group of the formula —O—alkyl). For example, methoxy, ethoxy, n-propoxy, sec-butoxy, t-butoxy, n-hexyloxy and the like. The term “halogenated alkoxy” shall mean any alkoxy group as defined above substituted with one to five halogen atoms, preferably with at least one halogen atom, preferably substituted with a least one fluoro atom. Suitable examples include but are not limited to —OCF₃, —OCHF₂, —OCH₂—CF₃, —OCF₂—CF₂—CF₂—CF₃, and the like. Similarly, the term “fluorinated alkoxy” shall mean any alkoxy group as defined above substituted with one to five fluoro atoms, preferably with at least one fluoro atom, preferably substituted with a least one fluoro atom. Suitable examples include but are not limited to —OCF₃, —OCHF₂, —OCH₂—CF₃, —OCF₂—CF₂—CF₂—CF₃, and the like.

The term “acyl” shall mean an organic radical of the formula —C(O)—(C₁₋₆alkyl) wherein the C₁₋₆alkyl is any straight or branched chain alkyl as herein defined; the acyl group may be derived from an organic acid by removal of the hydroxyl. Suitable examples include but are not limited to acetyl, propionyl and the like.

The term “aryl” shall refer to unsubstituted carbocylic aromatic groups such as phenyl, naphthyl, and the like. The term “aralkyl” shall mean an -(alkyl)-(aryl), such as benzyl, phenethyl, and the like; preferably the aralkyl group is of the formula —(C₁₋₄alkyl)-(aryl).

The term “heteroaryl” shall denote any five or six membered monocyclic aromatic ring structure containing at least one heteroatom selected from the group consisting of O, N and S, optionally containing one to three additional heteroatoms independently selected from the group consisting of O, N and S; or a nine or ten membered bicyclic aromatic ring structure containing at least one heteroatom selected from the group consisting of O, N and S, optionally containing one to four additional heteroatoms independently selected from the group consisting of O, N and S. The heteroaryl group may be attached at any heteroatom or carbon atom of the ring such that the result is a stable structure. Unless otherwise noted, the heteroaryl group may be optionally substituted with one or more substituents as herein defined. Examples of suitable heteroaryl groups include, but are not limited to, pyrrolyl, furyl, thienyl, oxazolyl, imidazolyl, pyrazolyl, isoxazolyl, isothiazolyl, triazolyl, thiadiazolyl, pyridyl, pyridazinyl, pyrimidinyl, pyrazinyl, pyranyl, furazanyl, indolizinyl, indolyl, isoindolinyl, indazolyl, benzofuryl, benzothienyl, benzimidazolyl, benzothiazolyl, purinyl, quinolinyl, isoquinolinyl, isothiazolyl, cinnolinyl, phthalazinyl, quinazolinyl, quinoxalinyl, naphthyridinyl, pteridinyl, and the like. Preferred heteroaryl groups include, but are not limited to pyridyl, quinolinyl and isoquinolinyl.

Unless specified otherwise, it is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein. One skilled in the art will further recognize that substituents may be bound to any of the atoms of a particular group (including, but not limited to C, N or S atoms), provided that the substitution results in a stable structure and does not violate valence rules.

When a particular group is “substituted” (e.g., alkyl, cycloalkyl, aryl, heteroaryl, heterocycloalkyl, etc.), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

As used herein, the notation “*” shall denote the presence of a stereogenic center.

Some of the compounds of the present invention may have trans and cis isomers. In addition, where the processes for the preparation of the compounds according to the invention give rise to a mixture of stereoisomers, these isomers may be separated by conventional techniques such as preparative chromatography. The compounds may be prepared as a single enantiomer in racemic form, or as a mixture of some possible stereoisomers. The non-racemic forms may be obtained by either synthesis or resolution. The compounds may, for example, be resolved into their component enantiomers by standard techniques, such as the formation of diastereomeric pairs by salt formation. The compounds may also be resolved by covalent linkage to a chiral auxiliary, followed by chromatographic separation and/or crystallographic separation, and removal of the chiral auxiliary. Alternatively, the compounds may be resolved using chiral chromatography.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at a diastereomeric excess of greater than or equal to about 80%, more preferably, at a diastereomeric excess of greater than or equal to about 90%, more preferably still, at a diastereomeric excess of greater than or equal to about 95%, more preferably still, at a diastereomeric excess of greater than or equal to about 98%, most preferably, at a diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

Under standard nomenclature used throughout this disclosure, the terminal portion of the designated side chain is described first, followed by the adjacent functionality toward the point of attachment. Thus, for example, a “phenylC₁-C₆alkyl-aminocarbonyl-C₁-C₆alkyl” substituent refers to a group of the formula

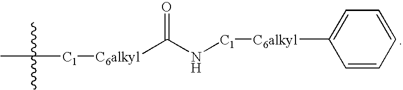

Abbreviations used in the specification, particularly the Schemes and Examples, are as follows:
Alloc=Allyloxycarbonyl
BOC or Boc=t-Butoxycarbonyl (i.e. —C(O)O—C(CH₃)₃)
n-BuLi=n-Butyl Lithium
Cbz=Benzyloxycarbonyl
DCE=Dichloroethane
DCM=Dichloromethane
DIPEA=Diisopropylethylamine
DME=1,2-Dimethoxyethane
DMSO=Dimethylsulfoxide
EtOAc=Ethyl Acetate
Etoc=Ethoxycarbonyl
EtOH=Ethanol
Fmoc=9-Fluorenylmethyloxycarbonyl
HPLC=High Performance Liquid Chromatography
MeCN=Acetonitrile
MeOH=Methanol
Mesylate=—O—SO₂—CH₃
MIC=Minimum Inhibitory Concentration
MPLC=Medium Pressure Liquid Chromatography
NaHMDS=Sodium bis(trimethylsilyl)amide
TDA-1=Tris(3,6-Dioxaheptyl)amine
TEA or Et₃N=Triethylamine
THF=Tetrahydrofuran
Tosylate=—O—SO₂-(4-methylphenyl)

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

The term “prophylactically effective amount” as used herein, means that amount of active compound or pharmaceutical agent that prevents the development of a condition, symptoms or manifestations thereof associated with bacterial infection. Thus it elicits the biological or medicinal response in a tissue system, animal or human that is being sought by a researcher, veterinarian, medical doctor or other clinician, which includes alleviation of the symptoms of the disease or disorder being treated.

The term “drug-resistant” or “drug-resistance” refers to the characteristics of a microbe to survive in the presence of a currently available antimicrobial agent such as an antibiotic at its routine, effective concentration.

As used herein, the term “composition” is intended to encompass a product comprising the specified ingredients in the specified amounts, as well as any product which results, directly or indirectly, from combinations of the specified ingredients in the specified amounts.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, the reaction step(s) is performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step. Further, one skilled in the art will recognize that wherein a reaction step of the present invention may be carried out in a variety of solvents or solvent systems, said reaction step may also be carried out in a mixture of the suitable solvents or solvent systems.

Examples of suitable solvents, bases, reaction temperatures, and other reaction parameters and components are provided in the detailed descriptions that follow herein. One skilled in the art will recognize that the listing of said examples is not intended, and should not be construed, as limiting in any way the invention set forth in the claims that follow thereafter.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any range therein.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

As used herein, unless otherwise noted, the term “oxygen protecting group” shall mean a group which may be attached to a oxygen atom to protect said oxygen atom from participating in a reaction and which may be readily removed following the reaction. Suitable oxygen protecting groups include, but are not limited to, acetyl, benzoyl, t-butyl-dimethylsilyl, trimethylsilyl (TMS), MOM, THP, and the like. Other suitable oxygen protecting groups may be found in texts such as T. W. Greene & P. G. M. Wuts, Protective Groups in Organic Synthesis, John Wiley & Sons, 1991.

As used herein, unless otherwise noted, the term “leaving group” shall mean a charged or uncharged atom or group that departs during a substitution or displacement reaction. Suitable examples include, but are not limited to, Br, Cl, I, triflate, tosylate, and the like.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

Additionally, chiral HPLC against a standard may be used to determine percent enantiomeric excess (%ee). The enantiomeric excess may be calculated as follows

[(Rmoles−Smoles)/(Rmoles+Smoles)]×100%

where Rmoles and Smoles are the R and S mole fractions in the mixture such that Rmoles+Smoles=1. The enantiomeric excess may alternatively be calculated from the specific rotations of the desired enantiomer and the prepared mixture as follows:

ee=([α−obs]/[α−max])×100.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laureate, malate, maleate, mandelate, mesylate, methylbromide, methylnitrate, methylsulfate, mucate, napsylate, nitrate, N-methylglucamine ammonium salt, oleate, pamoate(embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids that may be used in the preparation of pharmaceutically acceptable salts include, but are not limited to, the following: acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid and undecylenic acid.

Representative bases that may be used in the preparation of pharmaceutically acceptable salts include, but are not limited to, the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, sodium hydroxide, triethanolamine, tromethamine and zinc hydroxide.

The present invention includes within its scope prodrugs of the compounds of this invention. In general, such prodrugs will be functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

As used herein, unless otherwise noted, the term “isolated form” shall mean that the compound is present in a form which is separate from any solid mixture with another compound(s), solvent system or biological environment. In an embodiment, the compound of formula (I) is present in an isolated form.

As used herein, unless otherwise noted, the term “substantially pure form” shall mean that the mole percent of impurities in the isolated compound is less than about 5 mole percent, preferably less than about 2 mole percent, more preferably, less than about 0.5 mole percent, most preferably, less than about 0.1 mole percent. In an embodiment, the compound of formula (I) is present as a substantially pure form.

As used herein, unless otherwise noted, the term “substantially free of a corresponding salt form(s)” when used to described the compound of formula (I) shall mean that mole percent of the corresponding salt form(s) in the isolated base of formula (I) is less than about 5 mole percent, preferably less than about 2 mole percent, more preferably, less than about 0.5 mole percent, most preferably less than about 0.1 mole percent. In an embodiment, the compound of formula (I) is present as a form that is substantially free of corresponding salt forms.

In making the compounds of the invention, the order of synthetic steps may be varied to increase the yield of desired product. In addition, the skilled artisan will also recognize the judicious choice of reactions, solvents, and temperatures are an important component in successful synthesis. While the determination of optimal conditions, etc. is routine, it will be understood that a variety of compounds can be generated in a similar fashion, using the guidance of the schemes below.

It is further recognized that the skilled artisan in the art of organic chemistry can readily carry out standard manipulations of the organic compounds without further direction; that is, it is well within the scope and practice of the skilled artisan to carry out such manipulations. These include, but are not limited to, cycloadditions, oxidations, acylations, alkylations, esterifications and saponifications and the like. Examples of these manipulations are discussed in standard texts such as March, Advanced Organic Chemistry (Wiley), Carey and Sundberg, Advanced Organic Chemistry (Vol. 2), Feiser & Feiser, Reagents for Organic Synthesis (16 volumes), L. Paquette, Encyclopedia of Reagents for Organic Synthesis (8 volumes), Frost & Fleming, Comprehensive Organic Synthesis (9 volumes) and the like.

Additionally, the skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. Examples of these manipulations can be found for example in T. Greene, Protecting Groups in Organic Synthesis.

Starting materials used in preparing the compounds of the present invention are known, made by published synthetic methods or available from commercial vendors.

Compounds of formula (I) may be prepared according to the process outlined in Scheme 1.

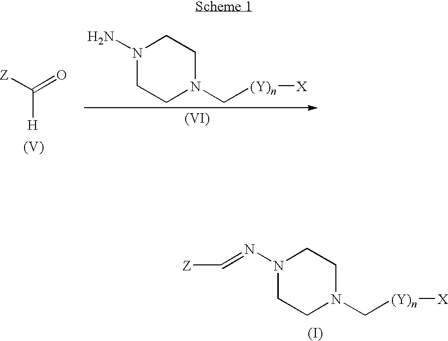

Accordingly, a suitably substituted compound of formula (V), a known compound or compound prepared by known methods, is reacted with a suitably substituted compound of formula (VI), a known compound or compound prepared by known methods, in an organic solvent such as THF, 1,4-dioxane, DME, DCM, DCE, and the like; preferably at a temperature in the range of from about 0° C. to about 60° C.; preferably for a period of time in the range of form about 30 minutes to about 24 hours; to yield the corresponding compound of formula (I).

Alternatively, compounds of formula (I) where X is selected from the group consisting of

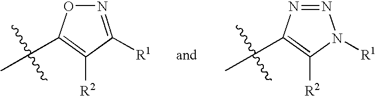

may alternatively be prepared according to the process outlined in Scheme 2.

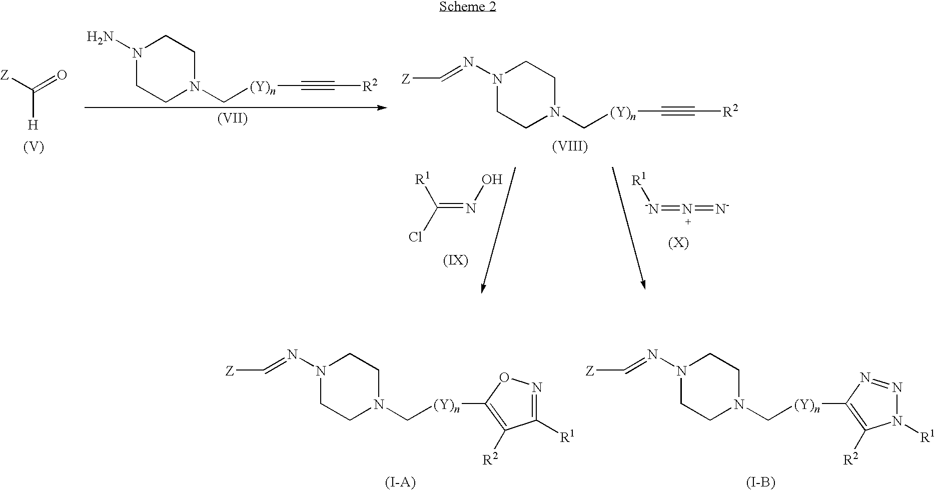

Accordingly, a suitably substituted compound of formula (V), a known compound or compound prepared by known methods is reacted with a suitably substituted compound of formula (VII), a known compound or compound prepared by known methods, in an organic solvent such as THF, 1,4-dioxane, DME, DCM, DCE, and the like; preferably at a temperature in the range of from about 0° C. to about 60° C.; preferably for a period of time in the range of form about 30 minutes to about 24 hours; to yield the corresponding compound of formula (VIII).

The compound of formula (VIII) is reacted with a suitably substituted compound of formula (IX), a known compound or compound prepared by known methods, in the presence of an organic or inorganic base such as TEA, DIPEA, K₂CO₃, Na₂CO₃, and the like; in an organic solvent such as DCM, DCE, THF, ethyl acetate, and the like; preferably at a temperature in the range of from about 25° C. to about 100° C.; preferably, for a period of time in the range of from about 1 hour to about 48 hours; to yield the corresponding compound of formula (I-A).

Alternately, the compound of formula (VIII) is reacted with a suitably substituted compound of formula (X), a known compound or compound prepared by known methods; under known cycloaddition conditions (for example, as described in Org. Lett., 2004, 6, 3897), to yield the corresponding compound of formula (I-B).

Compounds of formula (V) may be prepared according to previously described literature procedures or modifications thereof. For example, compounds of formula (V) may prepared according to the procedures described in U.S. Pat. No. 4,174,320 (and references included therein): Gazz. Chim. Ital. 1989, 119, 585-588; Antimicrob. Agents Chemother 1966, 6, 352-8; and Helv. Chim. Acta, 1993, 76,1459.

Compounds of formula (VI) may be prepared according to known methods. Alternatively, compounds of formula (VI) may be prepared according to the procedures as outlined in Scheme 3 through Scheme 7, which follow herein; and further as exemplified in Intermediate Example 3 through Intermediate Example 24, Intermediate Example 26, Intermediate Example 28 and Intermediate Example 29, which follow herein.

Compounds of formula (VI) wherein X is

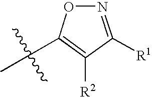

may be prepared according to the process outlined in Scheme 3.

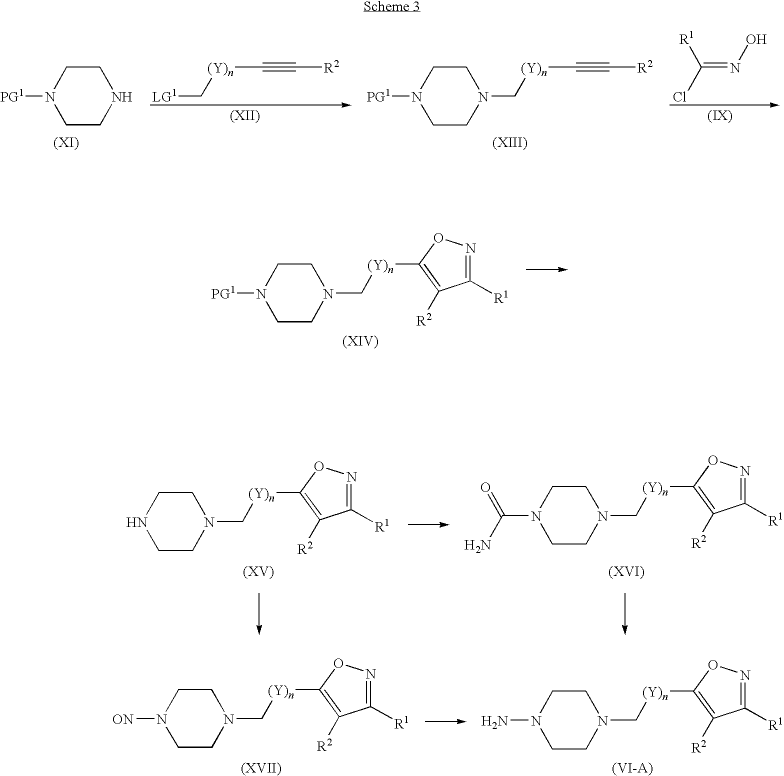

Accordingly, a suitably substituted compound of formula (XI), wherein PG¹ is a suitably selected nitrogen protecting group such as BOC, Cbz, benzyl, Fmoc, Etoc, Alloc, and the like, preferably BOC, a known compound or compound prepared by known methods, is reacted with a suitably substituted compound of formula (XII), wherein LG¹ is a suitably selected leaving group such as Br, Cl, I, tosylate, mesylate, and the like, preferably Br; in the presence of an organic or inorganic base such as TEA, DIPEA, K₂CO₃, Na₂CO₃, and the like; in an organic solvent such as DCM, DCE, THF, EtOAc, and the like; preferably at a temperature in the range of from about 25° C. to about 100° C.; preferably for a period of time in the range of from about 1 hour to about 48 hours; to yield the corresponding compound of formula (XIII).

The compound of formula (XIII) is reacted with a suitably substituted compound of formula (IX), a known compound or compound prepared by known methods; in the presence of an organic or inorganic base such as TEA, DIPEA, K₂CO₃, Na₂CO₃, and the like; in an organic solvent such as DCM, DCE, THF, EtOAc, and the like; preferably at a temperature in the range of from about 25° C. to about 100° C.; preferably for a period of time in the range of from about 1 hour to about 48 hours; to yield the corresponding compound of formula (XIV).

The compound of formula (XIV) is de-protected according to known methods, to yield the corresponding compound of formula (XV).

The compound of formula (XV) is reacted with aqueous sodium cyanate, potassium cyanate, and the like; in an aqueous solution at about pH 3; preferably at a temperature in the range of from about 0° C. to about 60° C.; preferably for a period of time in the range of from about 1 hour to about 48 hours; to yield the corresponding compound of formula (XVI).

The compound of formula (XVI) is reacted according to a two step process: (a) first with a suitably selected hypochlorite reagent such as sodium hypochlorite, potassium hypochlorite, and the like; in the presence of aqueous sodium hydroxide, aqueous potassium hydroxide, and the like; in an organic solvent such as methanol, ethanol, isopropanol, and the like; preferably at a temperature in the range of from about −10° C. to about 50° C.; preferably for a period of time in the range of from about 1 hour to about 24 hours; and then (b) acidified, preferably to about pH 1, with a suitably selected acid such as HCl, H₂SO₄, and the like; to yield the corresponding compound of formula (VI-A).

Alternatively, the compound of formula (XV) is reacted with a suitably selected nitrite, such as isoamyl nitrite, aqueous sodium nitrite, aqueous potassium nitrite, and the like; in an organic solvent such as THF, ethyl acetate, DCM, and the like; preferably at a temperature in the range of from about 0° C. to about 50° C.; preferably for a period of time in the range of from about 1 hour to about 48 hours; to yield the corresponding compound of formula (XVII).

The compound of formula (XVII) is reacted with a suitably selected reducing agent such as sodium borohydride, Raney nickel, zinc/acetic acid, and the like; in an organic solvent such as methanol, ethanol, and the like; preferably at a temperature in the range of from about 0° C. to about 50° C.; preferably for a period of time in the range of from about 1 hour to about 48 hours; to yield the corresponding compound of formula (VI-A).

Compounds of formula (VI) wherein X is

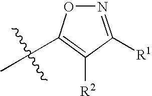

may alternatively be prepared according to the process outlined in Scheme 4.

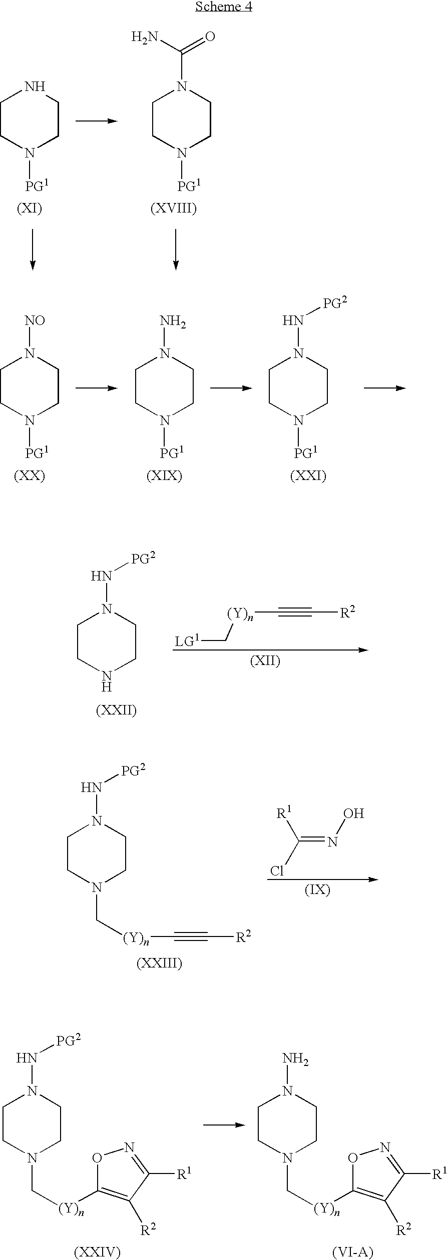

Accordingly, a suitably substituted compound of formula (Xi), wherein PG¹ is a suitably selected nitrogen protecting group such as BOC, Cbz, benzyl, Fmoc, Etoc, Alloc, and the like, preferably BOC, a known compound or compound prepared by known methods, is reacted with aqueous sodium cyanate, potassium cyanate, and the like; in an aqueous solution at about pH 3; preferably at a temperature in the range of from about 0° C. to about 60° C.; preferably for a period of time in the range of from about 1 hour to about 48 hours; to yield the corresponding compound of formula (XVIII).

The compound of formula (XVIII) is reacted according to a two step process: (a) first with a suitably selected hypochlorite reagent such as sodium hypochlorite, potassium hypochlorite, and the like; in the presence of aqueous sodium hydroxide, aqueous potassium hydroxide, and the like; in an organic solvent such as methanol, ethanol, isopropanol, and the like; preferably at a temperature in the range of from about −10° C. to about 50° C.; preferably for a period of time in the range of from about 1 hour to about 24 hours; and then (b) acidified, preferably to about pH 1, with a suitably selected acid such as HCl, H₂SO₄, and the like; to yield the corresponding compound of formula (XIX).

Alternatively, the compound of formula (Xl) is reacted with a suitably selected nitrite, such as isoamyl nitrite, aqueous sodium nitrite, aqueous potassium nitrite, and the like; in an organic solvent such as THF, ethyl acetate, DCM, and the like; preferably at a temperature in the range of from about 0° C. to about 50° C.; preferably for a period of time in the range of from about 1 hour to about 48 hours; to yield the corresponding compound of formula (XX).

The compound of formula (XX) is reacted with a suitably selected reducing agent such as sodium borohydride, Raney nickel, zinc/acetic acid, and the like; in an organic solvent such as methanol, ethanol, and the like; preferably at a temperature in the range of from about 0° C. to about 50° C.; preferably for a period of time in the range of from about 1 hour to about 48 hours; to yield the corresponding compound of formula (XIX).

The compound of formula (XIX) is protected with a suitably selected nitrogen protecting group (PG²), according to known methods, wherein the PG² nitrogen protecting group is preferably different from PG¹ and further, wherein the PG² nitrogen protecting group is selected such that it will not be removed under the conditions which remove the PG¹ group; to yield the corresponding compound of formula (XXI).

The compound of formula (XXI) is de-protected according to known methods to remove the PG¹ group, to yield the corresponding compound of formula (XXII).

The compound of formula (XXII) is reacted with a suitably substituted compound of formula (XII), wherein LG¹ is a suitably selected leaving group such as Br, Cl, I, tosylate, mesylate, and the like, preferably Br; in the presence of an organic or inorganic base such as TEA, DIPEA, K₂CO₃, Na₂CO₃, and the like; in an organic solvent such as DCM, DCE, THF, EtOAc, and the like; preferably at a temperature in the range of from about 25° C. to about 100° C.; preferably for a period of time in the range of from about 1 hour to about 48 hours; to yield the corresponding compound of formula (XXIII).

The compound of formula (XXIII) is reacted with a suitably substituted compound of formula (IX); in the presence of an organic or inorganic base such as TEA, DIPEA, K₂CO₃, Na₂CO₃, and the like; in an organic solvent such as DCM, DCE, THF, EtOAc, and the like; preferably at a temperature in the range of from about 25° C. to about 100° C.; preferably for a period of time in the range of from about 1 hour to about 48 hours; to yield the corresponding compound of formula (XXIV).

The compound of formula (XXIV) is de-protected according to known methods, to yield the corresponding compound of formula (VI-A).

Compounds of formula (VI) wherein X is

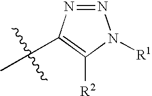

may be prepared according to the process outlined in Scheme 5.

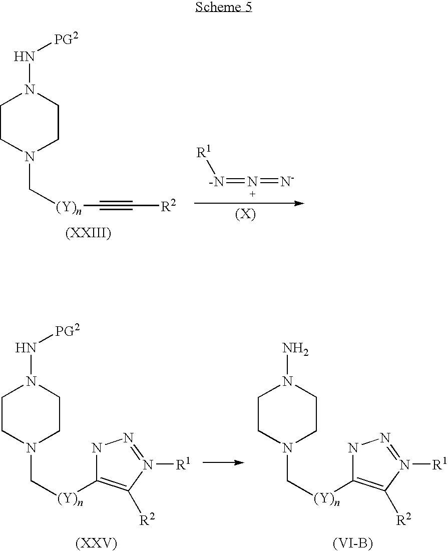

Accordingly, a suitably substituted compound of formula (XXIII), prepared as describe in for example, Scheme 4 above, is reacted with a suitably substituted compound of formula (X), a known compound or compound prepared by known methods, according to known cycloaddition methods (for example, as described in Org. Lett., 2004, 6, 3897), to yield the corresponding compound of formula (XXV). The compound of formula (XXV) is then deprotected according to known methods, to yield the corresponding compound of formula (VI-B).

Compounds of formula (VI), wherein X is

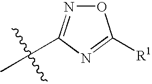

may be prepared according to the process outlined in Scheme 6.

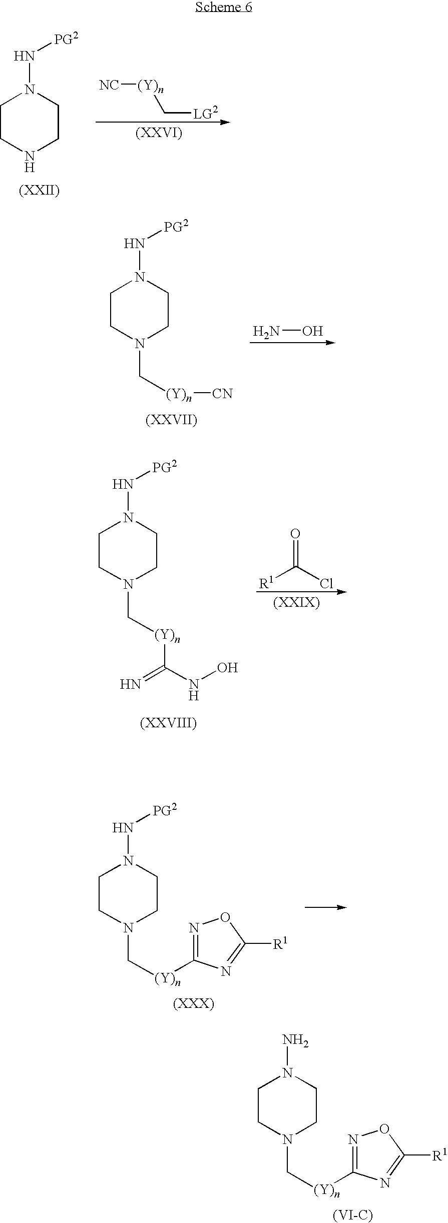

Accordingly, a suitably substituted compound of formula (XXII) is reacted with a suitably substituted compound of formula (XXVI), wherein LG² is a suitably selected leaving group such as Br, Cl, I, mesylate, tosylate, and the like, a known compound or compound prepared by known methods; in the presence of an organic or inorganic base such as TEA, DIPEA, Cs₂CO₃, K₂CO₃, Na₂CO₃, and the like; in an organic solvent such as THF, DCM, acetonitrile, and the like; preferably at a temperature in the range of from about 0° C. to about 50° C.; preferably for a period of time in the range of from about 1 hour to about 48 hours; to yield the corresponding compound of formula (XXVII).

The compound of formula (XXVII) is reacted with aqueous hydroxylamine; in an organic solvent such as methanol, ethanol, and the like; preferably at a temperature in the range of from about 0° C. to about 100° C.; preferably for a period of time in the range of from about 1 hour to about 48 hours; to yield the corresponding compound of formula (XXVIII).

The compound of formula (XXVIII) is reacted with a suitably selected acid chloride, a compound of formula (XXIX), a known compound or compound prepared by known methods; in the presence of an organic base such as pyridine, TEA, DIPEA, and the like; neat or in an organic solvent such as THF, DCM, and the like; preferably at a temperature in the range of from about 0° C. to about 100° C.; preferably for a period of time in the range of from about 1 hour to about 48 hours; to yield the corresponding compound of formula (XXX).

The compound of formula (XXX) is de-protected according to known methods, to yield the corresponding compound of formula (VI-C).

Compounds of formula (VI) may alternatively be prepared according to the process outlined in Scheme 7, below.

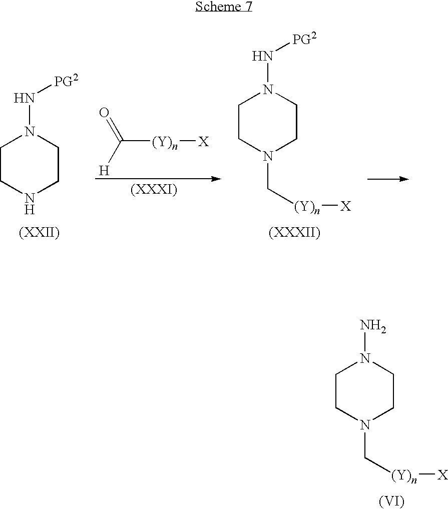

Accordingly, a suitably substituted compound of formula (XXII) is reacted with a suitably substituted compound of formula (XXXI), a known compound or compound prepared by known methods; in the presence of a suitably selected reducing agent such as sodium cyanoborohydride, sodium triacetoxyborohydride, and the like; in an organic solvent such as DCE, methanol, ethanol, and the like; preferably at a temperature in the range of from about 0° C. to about 50° C.; preferably for a period of time in the range of from about 1 hour to about 48 hours; to yield the corresponding compound of formula (XXXII).

The compound of formula (XXXII) is de-protected according to known methods, to yield the corresponding compound of formula (VI).

The present invention further comprises pharmaceutical compositions containing one or more compounds of formula (I) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gel caps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, though other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01-5000 mg or any range therein, and may be given at a dosage of from about 0.01-100 mg/kg/day, or any range therein, preferably from about 1 to about 50 mg/kg/day, or any range therein. The dosages, however, may be varied depending upon the requirement of the patients, the severity of the condition being treated and the compound being employed. The use of either daily administration or post-periodic dosing may be employed.

Preferably these compositions are in unit dosage forms such as tablets, pills, capsules, powders, granules, sterile parenteral solutions or suspensions, metered aerosol or liquid sprays, drops, ampoules, autoinjector devices or suppositories; for oral, parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.01 to about 1000 mg of the active ingredient of the present invention. The tablets or pills of the novel composition can be coated or otherwise compounded to provide a dosage form yielding the advantage of prolonged action. For example, the tablet or pill can comprise an inner dosage and an outer dosage component, the latter being in the form of an envelope over the former. The two components can be separated by an enteric layer, which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The methods described in the present invention may also be carried out using a pharmaceutical composition comprising any of the compounds as defined herein and a pharmaceutically acceptable carrier. The pharmaceutical composition may contain between about 0.01 mg and 1000 mg of the compound, or any range therein; preferably about 10 to 500 mg of the compound, or any range therein, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms may include suitably flavored suspending or dispersing agents such as the synthetic and natural gums, for example, tragacanth, acacia, methyl-cellulose and the like. For parenteral administration, sterile suspensions and solutions are desired. Isotonic preparations, which generally contain suitable preservatives, are employed when intravenous administration is desired.

The compound of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholine.

Compounds of the present invention may also be delivered by the use of monoclonal antibodies as individual carriers to which the compound molecules are coupled. The compounds of the present invention may also be coupled with soluble polymers as target able drug carriers. Such polymers can include polyvinylpyrrolidone, pyran copolymer, polyhydroxypropylmethacrylamidephenol, polyhydroxy-ethylaspartamidephenol, or polyethyleneoxidepolylysine substituted with palmitoyl residue. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

To prepare a pharmaceutical composition of the present invention, a compound of formula (I) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment with antimicrobial agents is required.

The daily dosage of the products may be varied over a wide range from 0.01 to 10,000 mg per adult human per day, or any range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250, 500 and 1000 milligrams of the active ingredient for the symptomatic adjustment of the dosage to the patient to be treated. An effective amount of the drug is ordinarily supplied at a dosage level of from about 0.01 mg/kg to about 100 mg/kg of body weight per day, or any range therein. Preferably, the range is from about 0.1 to about 50 mg/kg of body weight per day, or any range therein. More preferably, from about 0.5 to about 25 mg/kg of body weight per day, or any range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails including first-in-human, dose in the range of and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

In the Examples that follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### INTERMEDIATE EXAMPLE 1

### (4-Aminopiperazine-1-carboxylic acid tert-butyl ester), Compound 33

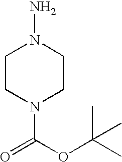

### Step A:

To N-Boc piperazine (10.0 g, 53.8 mmol) in 1N HCl (60 mL) was added 2N KOH dropwise until the pH of the resulting solution was about pH-3. Potassium cyanate (5.2 g, 64.5 mmol) was added and the resulting mixture was stirred at room temperature for 1 h. The resulting white precipitate was vacuum filtered to yield a white solid, which was used in the next step without further purification. MS 230 (M+1)⁺

### Step B:

The white solid from step A in ethanol (50 mL) was cooled to 0° C., and a pre-mixed solution of NaOCl (10-13% active chlorine, 43.4 mL) and 15% NaOH (78.0 mL) was added dropwise via an addition funnel. The ice bath was removed and the resulting mixture was stirred at room temperature for 1 h. 1N HCl was added to the resulting mixture until the pH of the solution was about pH 1, and the resulting mixture was stirred at room temperature for an additional 15 min. The pH of the solution was made basic with saturated aqueous potassium carbonate, and the resulting solution concentrated in vacuo to half the volume, then extracted three times with EtOAc, the combined organics were dried with Na₂SO₄, and concentrated in vacuo to yield the title compound, which was used in the next step without further purification.

MS 202 (M+1)⁺

### INTERMEDIATE EXAMPLE 2

### (4-Prop-2-ynyl-piperazin-1-yl)-carbamic acid ethyl ester, Compound 34

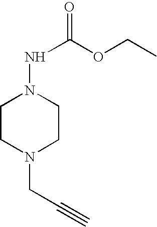

### Step A:

To Compound 33 prepared as in Intermediate Example 1 (8.29 g, 41.2 mmol) and pyridine (6.0 mL, 74.2 mmol) in MeCN (120 mL) was added dropwise ethyl chloroformate (5.9 mL, 61.9 mmol). The resulting mixture was stirred at room temperature for 3 h, then partitioned between EtOAc and saturated aqueous NaHCO₃, dried with Na₂SO₄, and concentrated in vacuo to yield a residue, which was used in the next step without further purification. MS 274 (M+1)⁺

### Step B:

To the residue from step A (9.64 g, 35.3 mmol), in DCM (50 mL) was added trifluoroacetic acid (5.0 mL, 67.3 mmol) and the resulting mixture was stirred at 60° C. for 6 h. Methanol (20 mL) was added and the resulting mixture was concentrated in vacuo to yield a residue, which was used in the next step without further purification. MS 174 (M+1)⁺

### Step C:

To a solution of the residue from step B and triethylamine (22.9 mL, 64.9 mmol) in acetone (120 mL) was added propargyl bromide (80% wt solution in toluene, 9.12 g, 61.9 mmol). The resulting mixture is heated at 60° C. for 2 h, and concentrated in vacuo. The resulting residue was partitioned between EtOAc and saturated aqueous NaHCO₃, dried with Na₂SO₄, and concentrated in vacuo. The resulting residue was purified by MPLC (SiO₂, 100% EtOAc) to yield the title compound.

MS 212 (M+1)⁺

### INTERMEDIATE EXAMPLE 3

### 4-(3-Phenyl-isoxazol-5-ylmethyl)-piperazin-1-ylamine, Compound 35

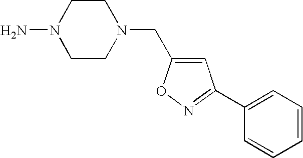

### Step A:

To a solution of compound 34 (540 mg, 2.56 mmol) and triethylamine (1.78 mL, 12.8 mmol) in DCM (3 mL) at room temperature was added dropwise a solution of alpha-chlorobenzaldoxime (2.0 g, 24.8 mmol) in DCM (7 mL). The resulting mixture was stirred at room temperature for 16 h, diluted with DCM, washed with saturated aqueous NaHCO₃, dried with Na₂SO₄, and concentrated in vacuo. The resulting residue was purified by MPLC (SiO₂, 2:1, hexanes:EtOAc) to yield a residue. MS 331 (M+1)⁺

### Step B:

To the residue from step A (446 mg, 1.34 mmol) in EtOH (10 mL), was added 3N NaOH (9 mL, 21.0 mmol), and the resulting mixture was heated at 100° C. until all starting material was consumed. The resulting mixture was extracted three times with EtOAc, the combined organic extracts were dried with Na₂SO₄, and concentrated in vacuo to yield the title compound, which was used in the next step without further purification.

MS 259 (M+1)⁺

### INTERMEDIATE EXAMPLE 4

### 4-[3-(4-Chloro-phenyl)-isoxazol-5-ylmethyl]-piperazin-1-ylamine, Compound 37

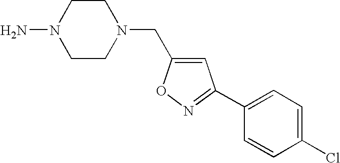

### Step A:

To a solution of Compound 36 (prepared as described in WO2003064413, 375 mg, 1.67 mmol) and triethylamine (0.35 mL, 2.51 mmol) in DCM (3 mL) at room temperature was added 4-chloro-N-hydroxy-benzenecarboximidoyl chloride (354 mg, 2.51 mmol) in small portions over 2 h. The resulting mixture was stirred overnight at room temperature, partitioned between EtOAc and saturated aqueous NaHCO₃, dried with Na₂SO₄, and concentrated in vacuo. The resulting residue was purified by MPLC (SiO₂, 2:1, hexanes:EtOAc) to yield a residue. MS 378 (M+1)⁺

### Step B:

To a solution of the residue from Step A (1.070 g, 2.84 mmol) in DCM (5 mL) was added trifluoroacetic acid (2.0 mL) and the resulting mixture was heated at 40° C. for 3 h. The resulting mixture was then diluted with DCM, washed with saturated aqueous NaHCO₃, dried with Na₂SO₄, and concentrated in vacuo to yield a residue, which was used in the next step without further purification. MS 278 (M+1)⁺

### Step C:

The residue from step B (606 mg, 2.18 mmol) was dissolved in 1N HCl (10 mL) and sufficient 2N KOH was added until the pH of the solution was pH3. Potassium cyanate (212 mg, 2.62 mmol) was added and the resulting mixture was stirred overnight. The resulting white precipitate was filtered and dried to yield a residue, which was used in the next step without further purification. MS 321 (M+1)⁺

### Step D:

A solution of the residue from step C (450 mg, 1.40 mmol) in EtOH (7 mL) was cooled to 0° C. and a pre-mixed solution of 15% NaOH (2.10 mL) and NaOCl (10-13% active chlorine, 1.20 mL) was added dropwise. The ice bath was removed and the resulting mixture was allowed to stir at room temperature until all starting material was consumed (1 h). 1N HCl was added until the solution was at pH 2, and the resulting mixture was stirred at room temperature for 10 min. Saturated aqueous potassium carbonate was added until the solution was basic and the resulting mixture was extracted three times with EtOAc, the combined organic extracts were dried with Na₂SO₄, and concentrated in vacuo to yield the title compound, which was used in the next step without further purification.

MS 293 (M+1)⁺

### INTERMEDIATE EXAMPLE 5

### 4-[3-(3-Chloro-phenyl)-isoxazol-5-ylmethyl]-Piperazin-1-ylamine, Compound 38

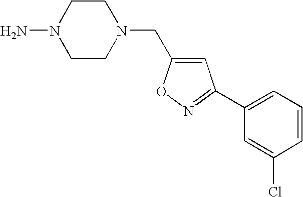

### Step A:

To a solution of compound 34 from Intermediate Example 2 (300 mg, 1.42 mmol) and triethylamine (1.97 mL, 14.16 mmol) in EtOAc (3 mL) at room temperature was added 3-chloro-N-hydroxybenzenecarboximidoyl chloride, (1.88 g, 10.0 mmol) in EtOAc (5.5 mL) via a syringe pump at an addition rate of 0.70 mL/h, and the resulting mixture stirred overnight. The resulting mixture was then partitioned between EtOAc and saturated aqueous NaHCO₃, dried with Na₂SO₄, and concentrated in vacuo. The resulting residue was purified by MPLC (SiO₂, 80-100% gradient elution, EtOAc % in Hexanes, to yield a residue. MS 365 (M+1)⁺

### Step B:

To the residue from step A (331 mg, 0.91 mmol) in EtOH (5 mL), was added 3N NaOH (1.81 mL, 5.45 mmol), and the resulting mixture was heated at 100° C. for 6 h. The resulting mixture was extracted three times with EtOAc, the combined organic extracts were dried with Na₂SO₄, and concentrated in vacuo to yield the title compound, which was used in the next step without further purification.

MS 293 (M+1)⁺

### INTERMEDIATE EXAMPLE 6

### 4-[3-(2,4-Dichloro-phenyl)-isoxazol-5-ylmethyl]-piperazin-1-ylamine, Compound 39

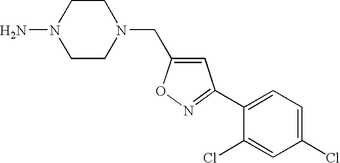

### Step A:

To a solution of compound 34 from Intermediate Example 2 (350 mg, 1.66 mmol) and triethylamine (2.30 mL, 16.53 mmol) in EtOAc (5 mL) at room temperature was added 2,4-dichloro-N-hydroxybenzenecarboximidoyl chloride, (3.09 g, 13.86 mmol) in EtOAc (8 mL) via a syringe pump at an addition rate of 1.5 mL/h, and the resulting mixture stirred overnight. The resulting mixture was partitioned between EtOAc and saturated aqueous NaHCO₃, dried with Na₂SO₄, and concentrated in vacuo. The resulting residue was purified by MPLC (SiO₂, 100% EtOAc), to yield a residue. MS 399 (M+1)⁺

### Step B:

To the residue from step A (250 mg, 0.63 mmol) in EtOH (1.2 mL), was added 3N NaOH (0.62 mL, 1.88 mmol), and the resulting mixture was heated at 100° C. for 6 h. The resulting mixture was extracted with EtOAc three times, the combined organic extracts were dried with Na₂SO₄, and concentrated in vacuo to yield the title compound, which was used in the next step without further purification.

MS 327 (M+1)⁺

### INTERMEDIATE EXAMPLE 7

### 4-[3-(4-Fluoro-phenyl)-isoxazol-5-ylmethyl]-piperazin-1-ylamine, Compound 40

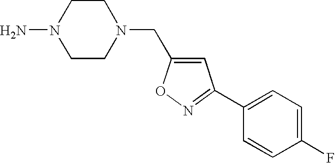

### Step A:

To a solution of compound 34 from Intermediate Example 2 (300 mg, 1.42 mmol) and triethylamine (1.97 mL, 14.2 mmol) in EtOAc (3 mL) at room temperature was added α-chloro-4-fluorobenzaldoxime, (1.96 g, 11.37 mmol) in EtOAc (5 mL) via a syringe pump at an addition rate of 1 mL/h, and the resulting mixture stirred overnight. The resulting mixture was partitioned between EtOAc and saturated aqueous NaHCO₃, dried with Na₂SO₄, and concentrated in vacuo. The resulting residue was purified by MPLC (SiO₂, 100% EtOAc) to yield a residue. MS 349 (M+1)⁺

### Step B:

To the residue from step A (286 mg, 0.82 mmol) in EtOH (5 mL), was added 3N NaOH (0.82 mL, 2.46 mmol), and the resulting mixture was heated at 100° C. for 6 h. The resulting mixture was extracted with EtOAc three times, the combined organic extracts were dried with Na₂SO₄, and concentrated in vacuo to yield the title compound, which was used in the next step without further purification.

MS 277 (M+1)⁺

### INTERMEDIATE EXAMPLE 8

### 4-[3-(3,4-Difluoro-phenyl)-isoxazol-5-ylmethyl]-piperazin-1-ylamine, Compound 41

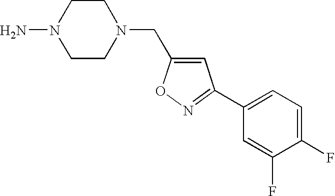

### Step A:

To a solution of compound 34 from Intermediate Example 2 (300 mg, 1.42 mmol) and triethylamine (1.97 mL, 14.16 mmol) in EtOAc (3 mL) at room temperature was added 3,4-difluoro-N-hydroxy-benzenecarboximidoyl chloride, (2.15 g, 11.20 mmol) in EtOAc (5 mL) via a syringe pump at an addition rate of 1 mL/h, and the resulting mixture stirred overnight. The resulting mixture was then partitioned between EtOAc and saturated aqueous NaHCO₃, dried with Na₂SO₄, and concentrated in vacuo. The resulting residue was purified by MPLC (SiO₂, 75-100% gradient elution, EtOAc % in hexanes, to yield a residue. MS 367(M+1)⁺

### Step B:

To the residue from step A (266 mg, 0.73 mmol) in EtOH (5 mL), was added 3N NaOH (1.45 mL, 4.66 mmol), and the resulting mixture was heated at 100° C. for 6 h. The resulting mixture was extracted with EtOAc three times, the combined organic extracts were dried with Na₂SO₄, and concentrated in vacuo to yield the title compound, which was used in the next step without further purification.

MS 295 (M+1)⁺

### INTERMEDIATE EXAMPLE 9

### 4-[3-(4-Trifluoromethyl-phenyl)-isoxazol-5-ylmethyl]-piperazin-1-ylamine, Compound 42

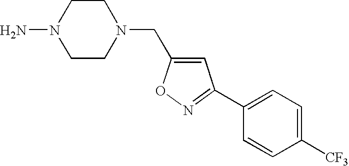

### Step A:

To a solution of compound 34 from Intermediate Example 2 (450 mg, 2.13 mmol) and triethylamine (2.37 mL, 17.0 mmol) in EtOAc (5 mL) at room temperature was added N-hydroxy-4-(trifluoromethyl)-benzenecarboximidoyl chloride, (1.78 g, 8.0 mmol) in EtOAc (5.5 mL) via a syringe pump at an addition rate of 0.70 mL/h, and the resulting mixture stirred overnight. The resulting mixture was then partitioned between EtOAc and saturated aqueous NaHCO₃, dried with Na₂SO₄, and concentrated in vacuo. The resulting residue was purified by MPLC (SiO₂, 100% EtOAc to yield a residue. MS 399 (M+1)⁺

### Step B

To the residue from step A (350 mg, 0.88 mmol) in EtOH (3 mL), was added 3N NaOH (0.9 mL, 2.64 mmol), and the resulting mixture was heated at 100° C. for 2 h. The resulting mixture was extracted with EtOAc three times, the combined organic extracts were dried with Na₂SO₄, and concentrated in vacuo to yield the title compound, which was used in the next step without further purification.

MS 327 (M+1)⁺

### INTERMEDIATE EXAMPLE 10

### 4-(3-p-Tolyl-isoxazol-5-ylmethyl)-piperazin-1-ylamine, Compound 43

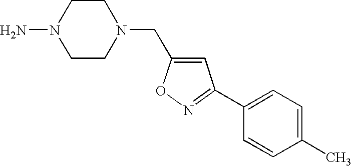

### Step A:

To a solution of compound 34 from Intermediate Example 2 (350 mg, 1.66 mmol) and triethylamine (2.95 mL, 21.2 mmol) in EtOAc (5 mL) at room temperature was added N-hydroxy-4-methyl-benzenecarboximidoyl chloride, (3.22 g, 19.05 mmol) in EtOAc (13.5 mL) via a syringe pump at an addition rate of 1 mL/h, and the resulting mixture stirred overnight. The resulting mixture was then partitioned between EtOAc and saturated aqueous NaHCO₃, dried with Na₂SO₄, and concentrated in vacuo. The resulting residue was purified by MPLC (SiO₂, 75-100% gradient elution, EtOAc % in Hexanes, to yield a residue. MS 345 (M+1)⁺

### Step B:

To the residue from step A (250 mg, 0.73 mmol) in EtOH (2 mL), was added 3N NaOH (0.73 mL, 2.18 mmol), and the resulting mixture was heated at 100° C. for 4 h. The resulting mixture was extracted with EtOAc three times, the combined organic extracts were dried with Na₂SO₄, and concentrated in vacuo to yield the title compound, which was used in the next step without further purification.

MS 273 (M+1)⁺

### INTERMEDIATE EXAMPLE 11

### 4-[3-(4-Methoxy-phenyl)-isoxazol-5-ylmethyl]-piperazin-1-ylamine, Compound 44

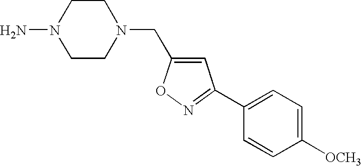

### Step A:

To a solution of compound 34 from Intermediate Example 2 (300 mg, 1.42 mmol) and triethylamine (1.18 mL, 8.49 mmol) in EtOAc (10 mL) at room temperature was added N-hydroxy-4-methoxybenzenecarboximidoyl chloride, (832 mg, 4.50 mmol) in small portions over 2 h, and the resulting mixture stirred overnight. The resulting mixture was then partitioned between EtOAc and saturated aqueous NaHCO₃, dried with Na₂SO₄, and concentrated in vacuo. The resulting residue was purified by MPLC (SiO₂, 100% EtOAc) to yield a residue. MS 361 (M+1)⁺

### Step B:

To the residue from step A (320 mg, 0.89 mmol) in EtOH (5 mL), was added 3N NaOH (0.30 mL, 0.90 mmol), and the resulting mixture was heated at 80° C. for 4 h. The resulting mixture was extracted with EtOAc three times, the combined organic extracts were dried with Na₂SO₄, and concentrated in vacuo to yield the title compound, which was used in the next step without further purification.

MS 289 (M+1)⁺

### INTERMEDIATE EXAMPLE 12

### 4-(3-Methyl-isoxazol-5-ylmethyl)-piperazin-1-ylamine, Compound 45

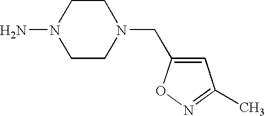

### Step A:

To a solution of compound 34 from Intermediate Example 2 (500 mg, 2.36 mmol) and triethylamine (9.84 mL, 70.8 mmol) in EtOAc (5 mL) at room temperature was added N-hydroxy-ethanimidoyl chloride, (3.0 g, 32.25 mmol) in EtOAc (5 mL) via a syringe pump at an addition rate of 1 mL/h, and the resulting mixture stirred overnight. The resulting mixture was then partitioned between EtOAc and saturated aqueous NaHCO₃, dried with Na₂SO₄, and concentrated in vacuo. The resulting residue was purified by MPLC (SiO₂, 100% EtOAc) to yield a residue. MS 269 (M+1)⁺

### Step B:

To the residue from step A (100 mg, 0.37 mmol) in EtOH (4 mL), was added 3N NaOH (1.0 mL, 3.0 mmol), and the resulting mixture was heated at 80° C. for 5 h. The resulting mixture was extracted with EtOAc three times, the combined organic extracts were dried with Na₂SO₄, and concentrated in vacuo to yield the title compound, which was used in the next step without further purification.

MS 197 (M+1)⁺

### INTERMEDIATE EXAMPLE 13

### [5-(4-Amino-piperazin-1-ylmethyl)-isoxazol-3-yl]-methanol, Compound 46

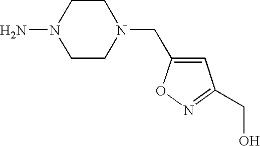

### Step A:

To a solution of compound 34 from Intermediate Example 2 (300 mg, 1.41 mmol) and triethylamine (3.92 mL, 28.2 mmol) in EtOAc (5 mL) at 75° C. was added ethyl chloroximido acetate (3.21 g, 21.2 mmol) in small portions over 6 h, and the resulting mixture stirred overnight. The resulting mixture was then partitioned between EtOAc and saturated aqueous NaHCO₃, dried with Na₂SO₄, and concentrated in vacuo. The resulting residue was purified by MPLC (SiO₂, 10% MeOH in DCM) to yield a residue. MS 327 (M+1)⁺

### Step B:

To the residue from step A (96 mg, 0.29 mmol) in ethanol (2 mL) at room temperature was added sodium borohydride (44 mg, 1.16 mmol). The resulting mixture was stirred at room temperature until all starting material was consumed. The reaction was then quenched with saturated aqueous NH₄Cl, the resulting mixture extracted with EtOAc, dried with Na₂SO₄, and concentrated in vacuo to yield a residue, which was used in the next step without further purification.

### Step C:

The residue from step C was diluted in EtOH (2 mL), 3N NaOH (0.3 mL, 0.90 mmol) was added and heated at 80° C. for 3 h. The resulting mixture was neutralized by the dropwise addition of 1N HCl, and concentrated in vacuo to yield the title compound, which was used in the next step without further purification.

MS 213 (M+1)⁺

### INTERMEDIATE EXAMPLE 14

### 4-(3-Dimethylaminomethyl-isoxazol-5-ylmethyl)-piperazin-1-ylamine, Compound 47

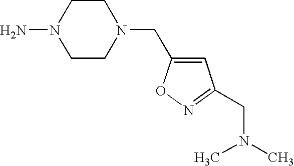

### Step A:

To a solution of compound 46 from Intermediate Example 13 (284 mg, 1.0 mmol) and triethylamine (0.28 mL, 2.0 mmol) in DCM (4 mL) was added methanesulfonyl chloride (0.12 mL, 1.5 mmol). The resulting mixture was stirred at room temperature for 2 h, diluted with DCM, washed with saturated aqueous NaHCO₃, dried with Na₂SO₄, and concentrated in vacuo. To the resultant residue in EtOH (2 mL) was added dimethylamine (40 wt % solution in water, 0.50 mL, 10 mmol) and the resulting mixture was heated at 80° C. overnight. The resulting mixture was concentrated in vacuo, diluted with EtOAc, washed with saturated aqueous NaHCO₃, dried with Na₂SO₄, and concentrated in vacuo to yield a residue. MS 312 (M+1)⁺

### Step B:

To the residue from step A (80 mg, 0.26 mmol) in EtOH (1 mL), was added 3N NaOH (1.0 mL, 3.0 mmol), and the resulting mixture was heated at 80° C. for 5 h. The resulting mixture was extracted with EtOAc three times, the combined organic extracts were dried with Na₂SO₄, and concentrated in vacuo to yield the title compound, which was used in the next step without further purification.

MS 240 (M+1)⁺

### INTERMEDIATE EXAMPLE 15

### 4-(3-Pyridin-2-yl-isoxazol-5-ylmethyl)-piperazin-1-ylamine, Compound 47

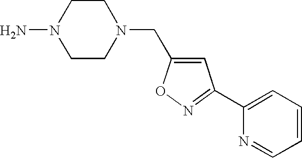

### Step A:

To a solution of compound 36 (prepared as described in WO 2003064413,1.35 g, 6.03 mmol) and triethylamine (4.16 mL, 29.90 mmol) in EtOAc (20 mL) at room temperature was added N-hydroxy-2-pyridinecarboximidoyl chloride, (2.16 g, 13.84 mmol) in small portions over 2 h. The resulting mixture was stirred at room temperature for an additional 2 h, and partitioned between EtOAc and saturated aqueous NaHCO₃, dried with Na₂SO₄, and concentrated in vacuo. The resulting residue was purified by MPLC (SiO₂, 1:2 Hexanes:EtOAc), then recrystallized from EtOAc to yield a residue. MS 345 (M+1)⁺

### Step B:

To the residue from step A (980 mg, 2.85 mmol) in DCM (10 mL) was added trifluoroacetic acid (2 mL). The resulting mixture was heated at 60° C. for 4 h, quenched with saturated aqueous NaHCO₃, extracted with EtOAc, dried with Na₂SO₄, and concentrated in vacuo to yield a residue, which was used in the next step without further purification.

### Step C:

The residue from step B was dissolved in 1N HCl (15 mL) and sufficient 2N KOH was added until the pH of the solution was pH 3. Potassium cyanate (346 mg, 4.27 g) was added and the resulting mixture was stirred overnight. The resulting mixture was then extracted three times with EtOAc, and the combined organics were dried with Na₂SO₄ and concentrated in vacuo to yield a residue, which was used in the next step without further purification. MS 288 (M+1)⁺

### Step D:

To the residue from step C (480 mg, 1.67 mmol) in EtOH (5 mL) at 0° C. was added dropwise a pre-mixed solution of 15% NaOH (2.5 mL) and NaOCl (10-13% active chlorine, 1.43 mL). The ice bath was removed and the resulting mixture was allowed to stir at room temperature for 1 h. 1N HCl was added to this resulting mixture until the pH of the solution was pH 1, and the resulting mixture was then stirred at room temperature for an additional 15 min. The pH of the solution was made basic with saturated aqueous potassium carbonate, and the resulting solution concentrated in vacuo to half the volume, then extracted three times with EtOAc. The combined organic extracts were dried with Na₂SO₄, and concentrated in vacuo to yield the title compound, which was used without further purification.

MS 260 (M+1)⁺

### INTERMEDIATE EXAMPLE 16

### 4-(3-Pyridin-3-yl-isoxazol-5-ylmethyl)-piperazin-1-ylamine, Compound 49

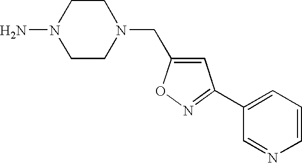

### Step A:

To a solution of compound 36 (prepared as described in WO 2003064413, 1.99 g, 8.86 mmol) and triethylamine (6.16 mL, 44.28 mmol) in EtOAc (40 mL) at room temperature was added N-hydroxy-3-pyridinecarboximidoyl chloride, (6.50 g, 41.67 mmol) in small portions over 4 h. The resulting mixture was stirred at room temperature overnight, partitioned between EtOAc and saturated aqueous NaHCO₃, dried with Na₂SO₄, and concentrated in vacuo. The resulting residue was recrystallized from EtOAc to yield a residue. MS 345 (M+1)⁺

### Step B:

To residue from step A (2.37 g, 6.89 mmol) in DCM (5 mL) was added trifluoroacetic acid (3 mL). The resulting mixture was heated at 60° C. for 4 h. MeOH (20 mL) was added and the resulting mixture was concentrated in vacuo to yield a residue, which was used in the next step without further purification.

### Step C:

The residue from step B was dissolved in 1N HCl (25 mL) and sufficient 2N KOH was added until the pH of the solution was pH 3. Potassium cyanate (669 mg, 8.27 g) was added and the resulting mixture was stirred overnight. The resulting mixture was extracted three times with EtOAc, and the combined organic extracts were dried with Na₂SO₄ and concentrated in vacuo. The resulting residue was recrystallized from isopropanol yield a residue. MS 288 (M+1)⁺

### Step D:

To the residue from step C (140 mg, 0.49 mmol) in EtOH (2 mL) at 0° C. was added dropwise a pre-mixed solution of 15% NaOH (0.71 mL) and NaOCl (10-13% active chlorine, 0.40 mL). The ice bath was removed and the resulting mixture was allowed to stir at room temperature for 1 h. 1N HCl was then added to the resulting mixture until the pH of the solution was pH 1, and the resulting mixture was then stirred at room temperature for an additional 5 min. The pH of the solution was made basic with saturated aqueous potassium carbonate, and the solution concentrated in vacuo to half its volume, then extracted three times with EtOAc. The combined organic extracts were dried with Na₂SO₄, and concentrated in vacuo to yield the title compound, which was used without further purification.

MS 260 (M+1)⁺

### INTERMEDIATE EXAMPLE 17

### 4-(3-Pyridin-4-yl-isoxazol-5-ylmethyl)-piperazin-1-ylamine, Compound 50

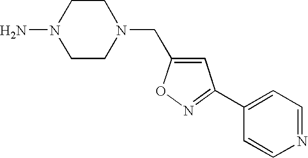

### Step A:

To a solution of compound 34 from Intermediate Example 2 (500 mg, 2.36 mmol) and triethylamine (2.62 mL, 18.8 mmol) in EtOAc (10 mL) at room temperature was added N-hydroxy-4-pyridinecarboximidoyl chloride (926 mg, 5.90 mmol) in small portions over 2 days. The resulting mixture was partitioned between EtOAc and saturated aqueous NaHCO₃, dried with Na₂SO₄, and concentrated in vacuo. The resulting residue was purified by MPLC (SiO₂, 8% MeOH in DCM) to yield a residue. MS 332 (M+1)⁺

### Step B:

To the residue from step A (450 mg, 1.35 mmol) in EtOH (5 mL), was added 3N NaOH (1.0 mL, 3.0 mmol), and the resulting mixture was heated at 100° C. for 6 h. The resulting mixture was extracted with EtOAc three times, the combined organic extracts were dried with Na₂SO₄, and concentrated in vacuo to yield the title compound, which was used in the next step without further purification.

MS 260 (M+1)⁺

### INTERMEDIATE EXAMPLE 18

### 4-{3-[4-(3-Dimethylamino-propoxy)-phenyl]-isoxazol-5-ylmethyl}-piperazin-1-ylamine, Compound 51

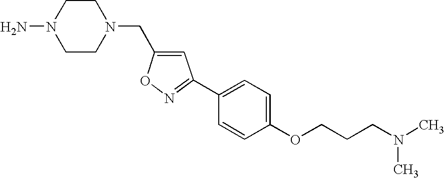

### Step A:

To a solution of compound 34 from Intermediate Example 2 (350 mg, 1.65 mmol) and triethylamine (1.84 mL, 13.26 mmol) in EtOAc (5.0 mL) at room temperature was added N-hydroxy-4-(3-dimethylamino-propoxy)-benzenecarboximidoyl chloride (2.11 g, 8.25 mmol) in small portions over 2 days. The resulting mixture was partitioned between EtOAc and saturated aqueous NaHCO₃, dried with Na₂SO₄, and concentrated in vacuo. The resulting residue was purified by MPLC (SiO₂, 10% MeOH in DCM) to yield a residue. MS 432 (M+1)⁺

### Step B:

To the residue from step A (358 mg, 0.83 mmol) in EtOH (2 mL), was added 3N NaOH (0.83 mL, 2.49 mmol), and the resulting mixture was heated at 100° C. for 3 h. The resulting mixture was extracted with EtOAc three times, the combined organic extracts were dried with Na₂SO₄, and concentrated in vacuo to yield the title compound, which was used in the next step without further purification.

MS 360 (M+1)⁺

### INTERMEDIATE EXAMPLE 19

### 5-(4-Amino-piperazin-1-ylmethyl)-37-(4-chloro-phenyl)-isoxazole-4-carboxylic acid methyl ester, Compound 52

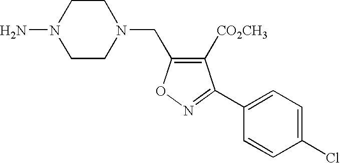

### Step A:

To a solution of compound 34 from Intermediate Example 2 (500 mg, 2.36 mmol) in THF (10 mL) at −78° C. was added n-BuLi (1.5 M in hexanes, 4.70 mL, 7.11 mmol) dropwise, and the resulting mixture was stirred at −78° C. for 30 min. Methyl chloroformate (0.55 mL, 7.11 mmol) was added, the resulting mixture was stirred at −78° C. for an additional 45 min, then quenched with saturated aqueous NaHCO₃, dried with Na₂SO₄, and concentrated in vacuo. The resulting residue was purified by MPLC (SiO₂, 1-5% gradient elution, MeOH % in DCM) to yield a residue. MS 270 (M+1)⁺

### Step B:

To the residue from step A (380 mg, 1.41 mmol) and triethylamine (1.57 mL, 11.30 mmol) in EtOAc (5.0 mL) at room temperature was added 4-chloro-N-hydroxy-benzenecarboximidoyl chloride, (1.19 g, 8.46 mmol) in small portions over two days. The resulting mixture was partitioned between EtOAc and saturated aqueous NaHCO₃, dried with Na₂SO₄, and concentrated in vacuo. The resulting residue was purified by MPLC (SiO₂, 100% EtOAc) to yield a residue. MS 423 (M+1)⁺

### Step C:

To the residue from step B (50 mg, 0.12 mmol) in EtOH (1 mL), was added 3N NaOH (0.07 mL, 0.24 mmol), and the resulting mixture was heated at 100° C. for 6 h. The resulting mixture was concentrated in vacuo, the resulting residue was dissolved in MeOH, the resulting mixture acidified with a few drops of concentrated HCl, and then heated at 100° C. for 3 days. The resulting mixture was partitioned between EtOAc and saturated aqueous NaHCO₃, dried with Na₂SO₄, and concentrated in vacuo to yield the title compound, which was used in the next step without further purification.

MS 351 (M+1)⁺

### INTERMEDIATE EXAMPLE 20

### 5-(4-Amino-piperazin-1-ylmethyl)-3-(4-chloro-phenyl)-isoxazole-4-carboxylic acid, Compound 53

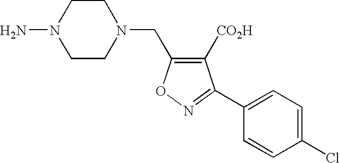

To the residue from step B, Intermediate Example 19 (280 mg, 0.66 mmol) in EtOH (2 mL) was added 3N NaOH (0.66 mL, 1.99 mmol), and the resulting mixture was heated at 100° C. for 4 h. The resulting mixture was concentrated in vacuo to dryness, the resulting residue dissolved in water (1 mL), and 1N HCl added to the resulting mixture until the pH of the solution was neutral to pH paper. The resulting mixture was then concentrated in vacuo, to yield the title compound as a residue, which was used in the next step without further purification. MS 337 (M+1)⁺

### INTERMEDIATE EXAMPLE 21

### 4-(5-Phenyl-thiophen-2-ylmethyl)-piperazin-1-ylamine, Compound 54

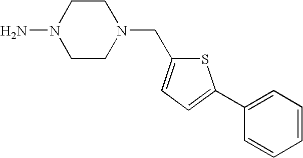

### Step A:

To the free base of 1-ethoxycarbonylaminopiperazine (prepared as described in step B in Intermediate Example 2, 730 mg, 4.22 mmol) and 5-phenyl-2-thiophenecarbaldehyde (793 mg, 4.22 mmol) in DCE was added acetic acid (0.48 mL, 8.44 mmol), and the resulting mixture was stirred at room temperature for 10 min. Sodium triacetoxyborohydride (1.78 g, 8.44 mmol) was then added and the resulting mixture was stirred at room temperature for 5 h. The resulting mixture was then quenched carefully with saturated aqueous NaHCO₃, extracted with DCM, dried with Na₂SO₄, and concentrated in vacuo. The resulting residue was purified by MPLC (SiO₂, 1-10% gradient elution, MeOH in DCM) to yield a residue. MS 346 (M+1)⁺

### Step B:

To residue from step A (534 mg, 1.55 mmol) in EtOH (5 mL), was added 3N NaOH (1.55 mL, 4.64 mmol), and the resulting mixture was heated at 100° C. for 5 h. The resulting mixture was extracted with EtOAc three times, the combined organic extracts were dried with Na₂SO₄, and concentrated in vacuo to yield the title compound, which was used in the next step without further purification.

MS 274 (M+1)⁺

### INTERMEDIATE EXAMPLE 22

### 4-[5-(4-Chloro-phenyl)-thiophen-2-ylmethyl]-piperazin-1-ylamine, Compound 55

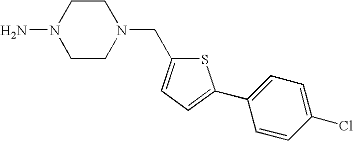

### Step A:

To the free base of 1-ethoxycarbonylaminopiperazine (prepared as described in step B in Intermediate Example 2, 1.17 g, 6.73 mmol) and 5-(4-chlorophenyl)-2-thiophenecarbaldehyde (1.0 g, 4.49 mmol) in DCE (15 mL) was added acetic acid (0.84 mL, 13.47 mmol), and sodium triacetoxyborohydride (2.37 g, 11.22 mmol). The resulting mixture was stirred at room temperature for 4 h, then quenched carefully with saturated aqueous NaHCO₃, extracted with DCM, dried with Na₂SO₄, and concentrated in vacuo. The resulting residue was purified by MPLC (SiO₂, 1-10% gradient elution, MeOH % in DCM) to yield a residue. MS 380 (M+1)⁺

### Step B:

To residue from Step A (330 mg, 0.87 mmol) in EtOH (1.6 mL), was added 3N NaOH (0.87 mL, 2.60 mmol), and the resulting mixture was heated at 100° C. for 4 h. The resulting mixture was extracted with EtOAc three times, the combined organic extracts were dried with Na₂SO₄, and concentrated in vacuo to yield the title compound, which was used in the next step without further purification. MS 308 (M+1)⁺

### INTERMEDIATE EXAMPLE 23

### 4-[5-(2-Chloro-phenyl)-furan-2-ylmethyl]-piperazin-1-ylamine, Compound 56

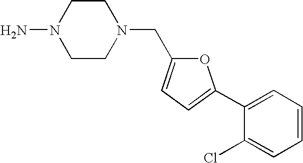

### Step A:

To the free base of 1-ethoxycarbonylaminopiperazine (prepared as described in step B in Intermediate Synthesis Example 2, 800 mg, 4.62 mmol) and 5-(2-chlorophenyl)-2-furancarboxaldehyde, (1.05 g, 5.08 mmol) in DCE (15 mL) was added acetic acid (0.48 mL, 8.38 mmol), and sodium triacetoxyborohydride (1.95 g, 9.24 mmol). The resulting mixture was stirred at room temperature overnight, then quenched carefully with saturated aqueous NaHCO₃, extracted with DCM, dried with Na₂SO₄, and concentrated in vacuo. The resulting residue was purified by MPLC (SiO₂, 1-10% gradient elution, MeOH % in DCM) to yield a residue. MS 364 (M+1)⁺

### Step B:

To the residue from step A (410 mg, 1.13 mmol) in EtOH (2 mL), was added 3N NaOH (1.13 mL, 3.38 mmol), and the resulting mixture was heated at 100° C. for 4 h. The resulting mixture was extracted with EtOAc three times, the combined organic extracts were dried with Na₂SO₄, and concentrated in vacuo to yield of the title compound, which was used in the next step without further purification.

MS 292 (M+1)⁺

### INTERMEDIATE EXAMPLE 24

### 4-[5-(4-Chloro-phenyl)-furan-2-ylmethyl]-piperazin-1-ylamine, Compound 57

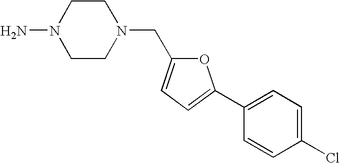

### Step A:

To the free base of 1-ethoxycarbonylaminopiperazine (prepared as described in step B in Intermediate Synthesis Example 2, 800 mg, 4.62 mmol) and 5-(4-chlorophenyl)-2-furancarboxaldehyde, (1.05 g, 5.08 mmol) in DCE (15 mL) was added acetic acid (0.48 mL, 8.38 mmol), and sodium triacetoxyborohydride (1.95 g, 9.24 mmol). The resulting mixture was stirred at room temperature overnight, quenched carefully with saturated aqueous NaHCO₃, extracted with DCM, dried with Na₂SO₄, and concentrated in vacuo. The resulting residue was purified by MPLC (SiO₂, 1-10% gradient elution, MeOH % in DCM) to yield a residue. MS 364 (M+1)⁺

### Step B:

To the residue from step A (410 mg, 1.13 mmol) in EtOH (2 mL), was added 3N NaOH (1.13 mL, 3.38 mmol), and the resulting mixture was heated at 100° C. for 24 h. The resulting mixture was extracted with EtOAc three times, the combined organic extracts were dried with Na₂SO₄, and concentrated in vacuo to yield the title compound, which was used in the next step without further purification.

MS 293 (M+1)⁺

### INTERMEDIATE EXAMPLE 25

### {4-[1-(4-Chloro-phenyl)-1H-[1,2,3]triazol-4-ylmethyl]-piperazin-1-yl}-carbamic acid ethyl ester, Compound 58

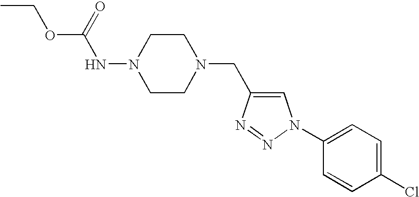

### Step A:

To a solution of compound 34 prepared as in Intermediate Example 2 (600 mg, 2.83 mmol) in DMSO (0.50 mL) and water (0.06 mL) was added 4-chloro-iodobenzene (674 mg, 2.83 mmol), L-proline (65 mg, 0.57 mmol), sodium carbonate (60 mg, 0.57 mmol), sodium azide (221 mg, 3.40 mmol), sodium ascorbate (55 mg, 0.20 mmol) and copper sulfate (35 mg, 0.14 mmol) and the resulting mixture was stirred at 65° C. overnight. The resulting mixture was then poured into water (20 mL). The resulting precipitate was filtered, the filter cake was washed with dilute ammonium hydroxide, air dried, and purified directly on MPLC (SiO₂, 1-10% gradient elution, MeOH % in DCM) to yield a residue. MS 366 (M+1)⁺

### Step B:

To the residue from Step A (300 mg, 0.82 mmol) in EtOH (3 mL), was added 3N NaOH (2.46 mL, 2.46 mmol), and the resulting mixture was heated at 100° C. for 4 h. The resulting mixture was partitioned between EtOAc and saturated aqueous NaHCO₃, dried with Na₂SO₄, and concentrated in vacuo to yield the title compound, which was used in the next step without further purification.

MS 293 (M+1)⁺

### INTERMEDIATE EXAMPLE 26

### 4-[5-(4-Chloro-phenyl)-[1,2,4]oxadiazol-3-ylmethyl]-piperazin-1-ylamine, Compound 59

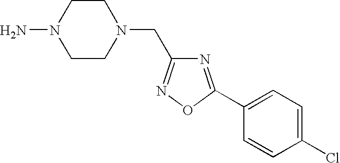

### Step A:

To 1-ethoxycarbonylaminopiperazine (prepared as described in step B in Intermediate Synthesis Example 2, 1.73 g, 10.0 mmol) in MeCN (20 mL) was added bromoacetonitrile (1.33 mL, 20 mmol) and potassium carbonate (8.28 g, 60 mmol). The resulting mixture was heated at 60° C. for 6 h, concentrated in vacuo, the residue partitioned between EtOAc and water, dried with Na₂SO₄, and concentrated in vacuo. The resulting residue was purified on MPLC (SiO₂, 100% EtOAc) to yield a residue. MS 213 (M+1)⁺

### Step B:

To the residue from step A (520 mg, 2.45 mmol) in EtOH (5 mL) was added hydroxylamine (50% solution in water, 0.30 mL, 3.68 mmol) and the resulting mixture was stirred at 80° C. for 3 h and concentrated in vacuo. To the resulting residue in pyridine (2.45 mL) was added 4-chloro-benzoyl chloride (875 mg, 5.0 mmol) and the resulting mixture was stirred at 80° C. for 6 h. The resulting mixture was then concentrated in vacuo, the resulting residue partitioned between EtOAc and saturated aqueous NaHCO₃, dried with Na₂SO₄, and concentrated in vacuo. The resulting residue was purified by MPLC (1-8% gradient elution, MeOH % in DCM) to yield a residue. MS 366 (M+1)⁺

### Step C:

To the residue from step B (64 mg, 0.17 mmol) in EtOH (1 mL), was added 3N NaOH (0.70 mL, 2.10 mmol), and the resulting mixture was heated at 95° C. for 6 h. The resulting mixture was extracted with EtOAc three times, the combined organic extracts were dried with Na₂SO₄, and concentrated in vacuo to yield the title compound, which was used in the next step without further purification.

MS 294 (M+1)⁺

### INTERMEDIATE EXAMPLE 27

### (4-{2-[5-(4-Chloro-phenyl)-isoxazol-3-yl]-ethyl}-piperazin-1-yl)-carbamic acid ethyl ester, Compound 60

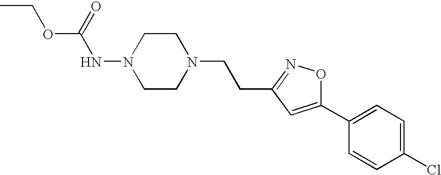

### Step A:

To [3-(4-chlorophenyl)-5-isoxazolyl]methanol (4.55 g, 21.8 mmol) in DCM (50 mL) was added Dess-Martin periodinane (10.15 g, 23.94 mmol) and the resulting mixture was stirred at room temperature for 1 h. The resulting mixture was then diluted with DCM, washed with 10% Na₂S₂O₃, saturated aqueous NaHCO₃, dried with Na₂SO₄, and concentrated in vacuo. The resulting residue was filtered through a bed of silica to yield a residue, which was used in the next step without further purification. MS 208 (M+1)⁺

### Step B:

To a suspension of (methoxymethyl)triphenylphosphonium chloride (3.11 g, 9.11 mmol) in THF (10 mL) at −10° C. was added NaHMDS (1 M in THF, 9.55 mL, 9.55 mmol) and the resulting mixture was stirred for 10 min. The residue from step A (943 mg, 4.55 mmol) was added and the resulting mixture stirred at −10° C. for an additional 2 h until all starting material was consumed. The reaction was quenched with saturated aqueous NH₄Cl, the resulting mixture was then extracted with EtOAc, dried with Na₂SO₄, and concentrated in vacuo. The resulting residue was purified by MPLC (5-30% gradient elution, EtOAc % in hexane) to yield a residue. MS 235 (M+1)⁺

### Step C:

To the residue from step B (535 mg, 2.29 mmol) in THF (5 mL) was added 1N HCl (5 mL) and the resulting mixture was stirred at 50° C. overnight. The resulting mixture was then partitioned between EtOAc and water, the organic layer dried with Na₂SO₄, and concentrated in vacuo to yield a residue, which was used in the next step without further purification.

### Step D:

To 1-ethoxycarbonylaminopiperazine (prepared as described in step B in Intermediate Example 2, 375 mg, 2.18 mmol) and the residue from step C above (400 mg, 1.81 mmol) in THF (1 mL) and EtOH (1 mL) was added sodium cyanoborohydride (456 mg, 7.24 mmol) and bromocresol green (˜2 mg). Acetic acid was then added to the resulting mixture until the color of the mixture turned from green to yellow, and the resulting mixture was then stirred at room temperature for 3 h, quenched with saturated aqueous NaHCO₃, extracted with EtOAc, dried with Na₂SO₄, and concentrated in vacuo. The resulting residue was purified by MPLC (1-5% gradient elution, MeOH % in DCM) to yield a residue. MS 379 (M+1)⁺

### Step E:

To the residue from step D (130 mg, 0.35 mmol) in EtOH (1 mL), was added 3N NaOH (0.35 mL, 1.03 mmol), and the resulting mixture was heated at 105° C. for 4 h. The resulting mixture was extracted with EtOAc three times, the combined organic extracts were dried with Na₂SO₄, and concentrated in vacuo to yield the title compound, which was used in the next step without further purification. MS 307 (M+1)⁺

### INTERMEDIATE EXAMPLE 28

### 4-[3-(3-Phenyl-isoxazol-5-yl)-propyl]-piperazin-1-ylamine, Compound 63

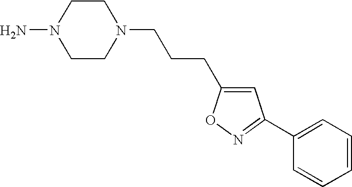

### and 4-{3-[3-(4-Chloro-phenyl)-isoxazol-5-yl]-propyl}-piperazin-1-ylamine, Compound 64

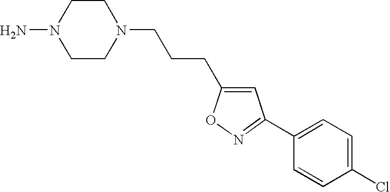

### Step A:

A mixture of 3-(4-chloro-phenyl)-isoxazole-5-carbaldehyde (prepared as described in step A, Intermediate Example 27, 1.23 g, 5.94 mmol), (1,3-dioxolan-2-ylmethyl)trimethylphosphonium bromide (3.82 g, 8.90 mmol) and TDA-1 (2.08 mL, 6.53 mmol), in saturated aqueous K₂CO₃ (15 mL) and DCM (15 mL) was heated to reflux overnight. The resulting mixture was diluted with DCM, the layers were partitioned and the organic layer was washed with saturated aqueous brine, dried with Na₂SO₄, and concentrated in vacuo. The resulting residue was dissolved in THF (15 mL) and 1N HCl (15 mL) and the resulting mixture stirred at room temperature for 1 h. The resulting mixture was then diluted with EtOAc, the layers partitioned, the organic layer washed with saturated aqueous NaHCO₃, dried with Na₂SO₄, and concentrated in vacuo to yield a residue, a 4:1 mixture of E:Z isomers of 3-[3-(4-chloro-phenyl)-isoxazol-5-yl]-propenal. MS 234 (M+1)⁺

### Step B:

To 1-ethoxycarbonylaminopiperazine (prepared as described in step B in Intermediate Example 2, 792 mg, 4.58 mmol) and the residue from step A (890 mg, 3.82 mmol) in THF (4 mL) and EtOH (1 mL) was added sodium cyanoborohydride (1.184 g, 19.1 mmol) and bromocresol green (˜2 mg). Acetic acid was added to the resulting mixture, until the color of the resulting mixture turned from green to yellow, and then the resulting mixture was stirred for 3 h. The resulting mixture was quenched with saturated aqueous NaHCO₃, extracted with EtOAc, the organic layer dried with Na₂SO₄, and concentrated in vacuo. The resulting residue was purified by MPLC (1-5% gradient elution, MeOH % in DCM) to yield a residue. MS 390 (M+1)⁺

### Step C:

The residue from step B (400 mg, 1.02 mmol) and 10% Pd/C (160 mg) in MeOH (2.5 mL) and EtOAc (2.5 mL) was hydrogenated in a Parr shaker at 30 psi of hydrogen for 30 min. The resulting mixture was filtered through a bed of CELITE®, and the resulting filtrate concentrated in vacuo to yield a residue, a 1:4 ratio of {4-[3-(3-Phenyl-isoxazol-5-yl)-propyl]-piperazin-1-yl}-carbamic acid ethyl ester MS 359 (M+1)⁺; and (4-{3-[3-(4-Chloro-phenyl)-isoxazol-5-yl]-propyl}-piperazin-1-yl)-carbamic acid ethyl ester MS 393 (M+1)⁺

### Step D:

To a mixture of the residue from step C above (180 mg, 0.46 mmol) in EtOH (0.5 mL), was added 3N NaOH (0.46 mL, 1.37 mmol), and the resulting mixture was heated at 100° C. for 6 h. The resulting mixture was partitioned between EtOAc and saturated aqueous NaHCO₃. The combined organic extracts were dried with Na₂SO₄, and concentrated in vacuo to yield a residue, a mixture of the title compounds, which mixture was used in the next step.

Compound 63: MS 287 (M+1)⁺;

Compound 64: MS 321 (M+1)⁺

### INTERMEDIATE EXAMPLE 29

### 4-[4-(4-Chloro-phenyl)-butyl]-piperazin-1-ylamine, Compound 65

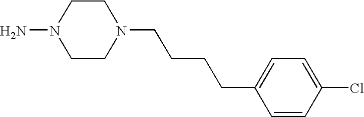

### Step A:

To 1-ethoxycarbonylaminopiperazine (prepared as described in step B in Intermediate Example 2, (512 mg, 2.96 mmol) and 4-(4-chloro-phenyl)-butyraldehyde (538 mg, 2.96 mmol) in DCE (10 mL) was added sodium triacetoxyborohydride (1.24 g, 5.92 mmol), and acetic acid (0.91 mL, 5.92 mmol) and the resulting mixture was stirred overnight at room temperature. The resulting mixture was then quenched with saturated aqueous NaHCO₃, extracted with EtOAc, dried with Na₂SO₄, and concentrated in vacuo. The resulting residue was purified by MPLC (1-8% gradient elution, MeOH % in DCM) to yield a residue. MS 340 (M+1)⁺

### Step B:

To the residue from step A (234 mg, 1.27 mmol) in EtOH (2 mL), was added 3N NaOH (2.54 mL, 7.63 mmol), and the resulting mixture was heated at 100° C. for 6 h. The resulting mixture was partitioned between EtOAc and saturated aqueous NaHCO₃, the combined organic extracts were dried with Na₂SO₄, and concentrated in vacuo to yield the title compound, which was used in the next step without further purification.

MS 307 (M+1)⁺

Unless otherwise noted, in the compounds prepared as described in Example 1 through Example 30, which follow herein, in the structures of the (RIF¹) and (RIF²) portions of the prepared compounds, the R⁶ group (as defined herein) is —C(O)—CH₃.

### SYNTHESIS EXAMPLES

### Representative Compounds of Formula (I)

### EXAMPLE 1

### Compound #1

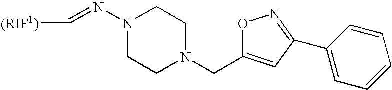

A solution of 3-formyl rifamycin SV (i.e. a compound of the formula (RIF¹)-C(O)H) (120 mg, 0.16 mmol) and 4-(3-phenyl-isoxazol-5-ylmethyl)-piperazin-1-ylamine (prepared as in Intermediate Example 3) (64 mg, 0.25 mmol) in THF (2 mL) was stirred at room temperature for 20 min. The resulting mixture was diluted with EtOAc, washed with a 5% solution of aqueous NaH₂PO₄ (˜pH 4), dried with Na₂SO₄, and concentrated in vacuo. The resulting residue was purified by MPLC (SiO₂, 1-5% gradient elution, MeOH % in DCM) to yield the title compound.

MS 966 (M+1)⁺

### EXAMPLE 2

### Compound #2

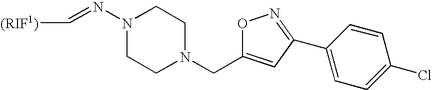

A solution of 3-formyl rifamycin SV (67 mg, 0.09 mmol) and 4-[3-(4-chloro-phenyl)-isoxazol-5-ylmethyl]-piperazin-1-ylamine (prepared as in Intermediate Example 4) (30 mg, 0.10 mmol) in THF (1 mL) was stirred at room temperature for 15 min. The resulting mixture was diluted with EtOAc, washed with a 5% solution of aqueous NaH₂PO₄ (˜pH 4), dried with Na₂SO₄, and concentrated in vacuo. The resulting residue was purified by MPLC (SiO₂, 1-5% gradient elution, MeOH % in DCM) to yield the title compound.

MS 1000 (M+1)⁺

### EXAMPLE 3

### Compound #3

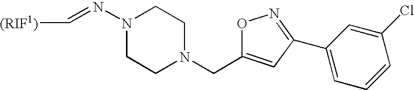

A solution of 3-formyl rifamycin SV (165 mg, 0.22 mmol) and 4-[3-(3-Chloro-phenyl)-isoxazol-5-ylmethyl]-piperazin-1-ylamine (prepared as in Intermediate Example 5) (200 mg, 0.68 mmol) in THF (2 mL) was stirred at room temperature for 1 h. The resulting mixture was diluted with EtOAc, washed with 5% aqueous NaH₂PO₄ (˜pH 4), dried with Na₂SO₄, and concentrated in vacuo. The resulting residue was purified by MPLC (SiO₂, 1-5% gradient elution, MeOH % in DCM) to yield the title compound.

MS 1000 (M+1)⁺

### EXAMPLE 4

### Compound #4

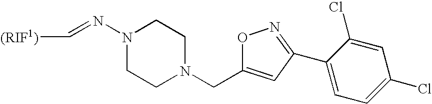

A solution of 3-formyl rifamycin SV (200 mg, 0.28 mmol) and 4-[3-(2,4-dichloro-phenyl)-isoxazol-5-ylmethyl]-piperazin-1-ylamine (prepared as in Intermediate Example 6) (180 mg, 0.55 mmol) in THF (2 mL) was stirred at room temperature for 1 h. The resulting mixture was diluted with EtOAc, washed with 5% aqueous NaH₂PO₄ (˜pH 4), dried with Na₂SO₄, and concentrated in vacuo. The resulting residue was purified by MPLC (SiO₂, 1-5% gradient elution, MeOH % in DCM) to yield the title compound.

MS 1034 (M+1)⁺

¹H NMR (400 MHz, CDCl₃): δ 8.30 (s, 1H), 7.71 (d, 1H), 7.52 (d, 1H), 7.35 (dd, 1H), 6.67 (s, 1H), 6.57 (m, 1H), 6.37 (d, 1H), 6.20 (d, 1H), 5.91 (dd, 1H), 5.10 (dd, 1H), 4.94 (d, 1H), 3.82 (s, 2H), 3.77 (m, 1H), 3.60 (m, 1H), 3.48 (m, 1H), 3.43 (s, 1H), 3.25-3.10 (m, 4H), 3.05 (s, 3H), 3.02-2.96 (m, 1H), 2.80-2.65 (m, 4H), 2.40-2.31 (m,1H), 2.24 (s, 3H), 2.07 (s, 3H), 2.06 (s, 3H), 1.80 (s, 3H), 1.73-1.64 (m, 1H), 1.58-1.52 (m, 1H), 1.39-1.32 (m, 1H), 1.01 (d, 3H), 0.85 (d, 3H), 0.60 (d, 3H), and −0.29 (d, 3H).

### EXAMPLE 5

### Compound #5

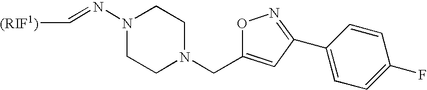

A solution of 3-formyl rifamycin SV (262 mg, 0.36 mmol) and 4-[3-(4-fluoro-phenyl)-isoxazol-5-ylmethyl]-piperazin-1-ylamine (prepared as in Intermediate Example 7) (200 mg, 0.73 mmol) in THF (2 mL) was stirred at room temperature for 1 h. The resulting mixture was diluted with EtOAc, washed with 5% aqueous NaH₂PO₄ (˜pH 4), dried with Na₂SO₄, and concentrated in vacuo. The resulting residue was purified by MPLC (SiO₂, 1-5% gradient elution, MeOH % in DCM) to yield the title compound.

MS 984 (M+1)⁺

### EXAMPLE 6

### Compound #6

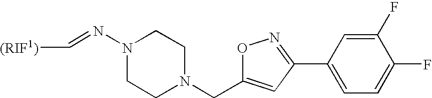

A solution of 3-formyl rifamycin SV (148 mg, 0.20 mmol) and 4-[3-(3,4-difluoro-phenyl)-isoxazol-5-ylmethyl]-piperazin-1-ylamine (prepared as in Intermediate Example 8) (181 mg, 0.61 mmol) in THF (1 mL) was stirred at room temperature for 1 h. The resulting mixture was diluted with EtOAc, washed with 5% aqueous NaH₂PO₄ (˜pH 4), dried with Na₂SO₄, and concentrated in vacuo. The resulting residue was purified by MPLC (SiO₂, 1-5% gradient elution, MeOH % in DCM) to yield the title compound.

MS 1002 (M+1)⁺

### EXAMPLE 7

### Compound #7

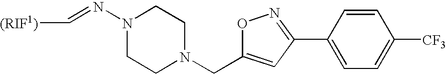

A solution of 3-formyl rifamycin SV (300 mg, 0.41 mmol) and 4-[3-(4-trifluoromethyl-phenyl)-isoxazol-5-ylmethyl]-piperazin-1-ylamine (prepared as in Intermediate Example 9) (269 mg, 0.82 mmol) in THF (2 mL) was stirred at room temperature for 1 h. The resulting mixture was diluted with EtOAc, washed with 5% aqueous NaH₂PO₄ (˜pH 4), dried with Na₂SO₄, and concentrated in vacuo. The resulting residue was purified by MPLC (SiO₂, 1-5% gradient elution, MeOH % in DCM) to yield the title compound.

MS 1034 (M+1)⁺

¹H NMR (400 MHz, CDCl₃): δ 8.29 (s,1H), 7.93 (d, 2H), 7.73 (d, 2H), 6.62-6.55 (m, 2H), 6.37 (d, 1H), 6.21 (d, 1H), 5.92 (dd, 1H), 5.10 (dd, 1H), 4.93 (d, 1H), 3.82-3.81 (m, 2H), 3.77 (d,1H), 3.64 (broad S,1H), 3.48-3.47 (m, 2H), 3.24-3.10 (m, 4H), 3.05 (s, 3H), 3.00 (m, 1H), 2.79-2.69 (m, 4H), 2.44-2.36 (m, 1H), 2.23 (s, 3H), 2.07 (s, 3H), 2.06 (s, 3H), 1.80 (s, 3H), 1.73-1.64 (m, 1H), 1.58-1.50 (m, 1H), 1.39-1.31 (m, 1H), 1.01 (d, 3H), 0.86 (d, 3H), 0.60 (d, 3H), and −0.30 (d, 3H).

### EXAMPLE 8

### Compound #8

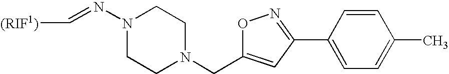

A solution of 3-formyl rifamycin SV (266 mg, 0.36 mmol) and 4-(3-p-tolyl-isoxazol-5-ylmethyl)-piperazin-1-ylamine (prepared as in Intermediate Example 10) (180 mg, 0.66 mmol) in THF (1 mL) was stirred at room temperature for 1 h. The resulting mixture was diluted with EtOAc, washed with 5% aqueous NaH₂PO₄ (˜pH 4), dried with Na₂SO₄, and concentrated in vacuo. The resulting residue was purified by MPLC (SiO₂, 1-5% gradient elution, MeOH % in DCM) to yield the title compound.

MS 980 (M+1)⁺

### EXAMPLE 9

### Compound #9

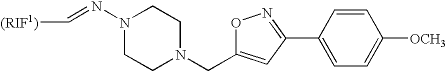

A solution of 3-formyl rifamycin SV (419 mg, 0.58 mmol) and 4-[3-(4-methoxy-phenyl)-isoxazol-5-ylmethyl]-piperazin-1-ylamine (prepared as in Intermediate Example 11) (200 mg, 0.69 mmol) in THF (1 mL) was stirred at room temperature for 1 h. The resulting mixture was diluted with EtOAc, washed with 5% aqueous NaH₂PO₄ (˜pH 4), dried with Na₂SO₄, and concentrated in vacuo. The resulting residue was purified by MPLC (SiO₂, 1-5% gradient elution, MeOH % in DCM) to yield the title compound. MS 996 (M+1)⁺

### EXAMPLE 10

### Compound #10

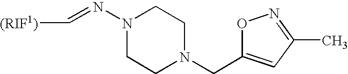

A solution of 3-formyl rifamycin SV (104 mg, 0.14 mmol) and 4-(3-methyl-isoxazol-5-ylmethyl )-piperazin-1-ylamine (prepared as in Intermediate Example 12) (50 mg, 0.25 mmol) in THF (1 mL) was stirred at room temperature for 1 h. The resulting mixture was diluted with EtOAc, washed with 5% aqueous NaH₂PO₄ (˜pH 4), dried with Na₂SO₄, and concentrated in vacuo. The resulting residue was purified by MPLC (SiO₂, 1-5% gradient elution, MeOH % in DCM) to yield the title compound.

MS 904 (M+1)

¹H NMR (400 MHz, CDCl₃): δ 8.27 (s, 1H), 6.57 (dd, 1H), 6.37 (d, 1H), 6.21 (d, 1H), 6.03 (s, 1H), 5.91 (dd, 1H), 5.10 (dd, 1H), 4.94 (d, 1H), 3.77 (d, 1H), 3.71-3.70 (s, 2H), 3.65-3.58 (broad S, 1H), 3.47 (d, 1H), 3.20-3.07 (m, 4H), 3.04 (s, 3H), 3.00 (m, 1H), 2.70-2.60 (m, 4H), 2.41-2.34 (m, 1H), 2.31 (s, 3H), 2.23 (s, 3H), 2.07 (s, 3H), 2.06 (s, 3H), 1.80 (s, 3H), 1.73-1.67 (m, 1H), 1.58-1.50 (m, 1H), 1.39-1.31 (m, 1H), 1.01 (d, 3H), 0.85 (d, 3H), 0.59 (d, 3H), and −0.30 (d, 3H).

### EXAMPLE 11

### Compound #11

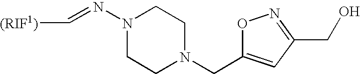

A solution of 3-formyl rifamycin SV (52 mg, 0.07 mmol) and [5-(4-amino-piperazin-1-ylmethyl)-isoxazol-3-yl]-methanol (prepared as in Intermediate Example 13) (15 mg, 0.07 mmol) in THF (1 mL) and DMSO (0.1 mL) was stirred at room temperature for 1 h. The resulting mixture was diluted with EtOAc, washed with 5% aqueous NaH₂PO₄ (˜pH 4), dried with Na₂SO₄, and concentrated in vacuo. The resulting residue was purified by MPLC (SiO₂, 1-5% gradient elution, MeOH % in DCM) to yield the title compound.

MS 920 (M+1)⁺

### EXAMPLE 12

### Compound #12

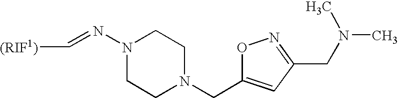

A solution of 3-formyl rifamycin SV (20 mg, 0.03 mmol) and 4-(3-dimethylaminomethyl-isoxazol-5-ylmethyl)-piperazin-1-ylamine (prepared as in Intermediate Example 14) (50 mg, 0.21 mmol) in THF (0.5 mL) was stirred at room temperature for 30 min. The resulting mixture was diluted with EtOAc, washed with 5% aqueous NaH₂PO₄ (˜pH 4), dried with Na₂SO₄, and concentrated in vacuo. The resulting residue was purified by HPLC (C-18, 20-60% gradient elution, MeCN % in water) to yield the title compound.

MS 947 (M+1)⁺

### EXAMPLE 13

### Compound #13

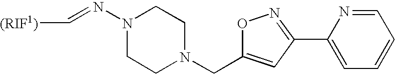

A solution of 3-formyl rifamycin SV (120 mg, 0.17 mmol) and 4-(3-pyridin-2-yl-isoxazol-5-ylmethyl)-piperazin-1-ylamine (prepared as in Intermediate Example 15) (110 mg, 0.42 mmol) in THF (1 mL) was stirred at room temperature for 1 h. The resulting mixture was diluted with EtOAc, washed with 5% aqueous NaH₂PO₄ (˜pH 4), dried with Na₂SO₄, and concentrated in vacuo. The resulting residue was purified by MPLC (SiO₂, 1-5% gradient elution, MeOH % in DCM) to yield the title compound. MS 967 (M+1)⁺

¹H NMR (400 MHz, CDCl₃): δ 8.69 (dd, 1H), 8.29 (s, 1H), 8.08 (d, 1H), 7.81 (ddd, 1H), 7.36 (ddd, 1H), 6.85 (s, 1H), 6.57 (dd, 1H), 6.35 (d, 1H), 6.20 (d, 1H), 5.91 (dd, 1H), 5.10 (dd, 1H), 4.94 (d, 1H), 3.83-3.82 (m, 2H), 3.75 (d, 1H), 3.60 (d, 1H), 3.47 (d, 1H), 3.42 (s, 1H), 3.24-3.07 (m, 4H), 3.04 (s, 3H), 3.03-2.97 (m, 1H), 2.78-2.66 (m, 4H), 2.39-2.31 (m, 1H), 2.23 (s, 3H), 2.07 (s, 3H), 2.06 (s, 3H), 1.80 (s, 3H), 1.75-1.65 (m, 1H), 1.59-1.50 (m, 1H), 1.41-1.31 (m, 1H), 1.00 (d, 3H), 0.85 (d, 3H), 0.59 (d, 3H), and -0.29 (d, 3H).

### EXAMPLE 14

### Compound #14

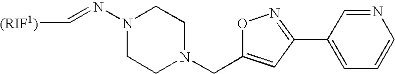

A solution of 3-formyl rifamycin SV (64 mg, 0.09 mmol) and 4-(3-pyridin-3-yl-isoxazol-5-ylmethyl)-piperazin-1-ylamine (prepared as in Intermediate Example 16) (30 mg, 0.12 mmol) in THF (1 mL) was stirred at room temperature for 1 h. The resulting mixture was diluted with EtOAc, washed with 5% aqueous NaH₂PO₄ (˜pH 4), dried with Na₂SO₄, and concentrated in vacuo. The resulting residue was purified by HPLC (C-18, 20-60% gradient elution, MeCN % in water) to yield the title compound. MS 967 (M+1)⁺

### EXAMPLE 15

### Compound #15

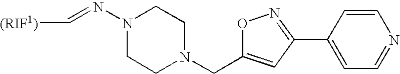

A solution of 3-formyl rifamycin SV (130 mg, 0.18 mmol) and 4-(3-pyridin-4-yl-isoxazol-5-ylmethyl)-piperazin-1-ylamine (prepared as in Intermediate Example 17) (60 mg, 0.23 mmol) in THF (1 mL) was stirred at room temperature for 1 h. The resulting mixture was diluted with EtOAc, washed with 5% aqueous NaH₂PO₄ (˜pH 4), dried with Na₂SO₄, and concentrated in vacuo. The resulting residue was purified by MPLC (SiO₂, 1-6% gradient elution, MeOH % in DCM) to yield the title compound.

MS 967 (M+1)⁺

### EXAMPLE 16

### Compound #16

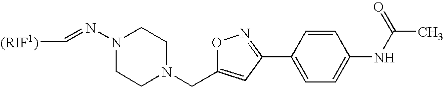

To a solution of a compound of the following structural formula

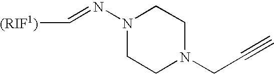

(preparation as described in U.S. Pat. No. 4,002,754), (75 mg, 0.09 mmol) and triethylamine (0.12 mL, 0.89 mmol) in EtOAc (0.5 mL) at room temperature was added 4-(acetylamino)-N-hydroxy-benzenecarboximidoyl chloride, (75 mg, 0.35 mmol) in small portions over 6 hours. The resulting mixture was partitioned between EtOAc, washed with 5% aqueous NaH₂PO₄, dried with Na₂SO₄, and concentrated in vacuo. The resulting residue was dissolved in a 1:1 solution of THF/H₂O; excess ascorbic acid was added and the resulting mixture stirred for 30 min. The resulting mixture was partitioned between EtOAc and water, dried with Na₂SO₄, and concentrated in vacuo. The resulting residue was purified by HPLC (C-18, 60-100% gradient elution, MeCN % in water) to yield the title compound.

MS 1023 (M+1)⁺

### EXAMPLE 17

### Compound #17

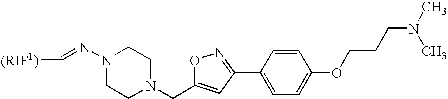

A solution of 3-formyl rifamycin SV (247 mg, 0.34 mmol) and 4-{3-[4-(3-dimethylamino-propoxy)-phenyl]-isoxazol-5-ylmethyl}-piperazin-1-ylamine (prepared as in Intermediate Example 18) (245 mg, 0.68 mmol) in THF (2 mL) was stirred at room temperature for 1 h. The resulting mixture was diluted with EtOAc, washed with 5% aqueous NaH₂PO₄ (˜pH 4), dried with Na₂SO₄, and concentrated in vacuo. The resulting residue was purified by MPLC (SiO₂, 1-5% gradient elution, MeOH % in DCM) to yield the title compound.

MS 1067 (M+1)⁺

### EXAMPLE 18

### Compound #18

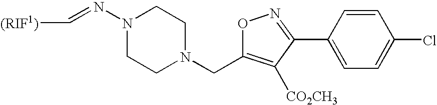

A solution of 3-formyl rifamycin SV (15 mg, 0.02 mmol) and 5-(4-amino-piperazin-1-ylmethyl)-3-(4-chloro-phenyl)-isoxazole-4-carboxylic acid methyl ester (prepared as in Intermediate Example 19) (15 mg, 0.04 mmol) in THF (0.5 mL) was stirred at room temperature for 5 h. The resulting mixture was stirred at room temperature for 5 h, diluted with EtOAc, washed with 5% aqueous NaH₂PO₄ (˜pH 4), dried with Na₂SO₄, and concentrated in vacuo. The resulting residue was purified by preparative TLC (SiO₂, 5% MeOH in DCM) to yield the title compound.

MS 1056 (M+1)⁺

### EXAMPLE 19

### Compound #19

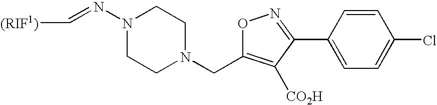

A solution of 3-formyl rifamycin SV (87 mg, 0.12 mmol) and 5-(4-amino-piperazin-1-ylmethyl)-3-(4-chloro-phenyl)-isoxazole-4-carboxylic acid (prepared as in Intermediate Example 20) (˜0.30 mmol) in THF (0.5 mL) was stirred at room temperature for 5 h. The resulting mixture was diluted with EtOAc, washed with 5% aqueous NaH₂PO₄ (˜pH 4), dried with Na₂SO₄, and concentrated in vacuo. The resulting residue was purified by HPLC (C-18, 80-100% gradient elution, MeCN % in water) to yield the title compound.

MS 1044 (M+1)⁺

### EXAMPLE 20

### Compound #20

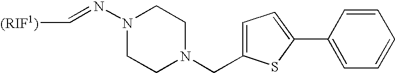

A solution of 3-formyl rifamycin SV (241 mg, 0.33 mmol) and 4-(5-phenyl-thiophen-2-ylmethyl)-piperazin-1-ylamine (prepared as in Intermediate Example 21) (273 mg, 1.0 mmol) in THF (2 mL) was stirred at room temperature for 2 h. The resulting mixture was diluted with EtOAc, washed with 5% aqueous NaH₂PO₄ (˜pH 4), dried with Na₂SO₄, and concentrated in vacuo. The resulting residue was purified by MPLC (SiO₂, 1-8% gradient elution, MeOH % in DCM) to yield the title compound.

MS 981 (M+1)⁺

### EXAMPLE 21

### Compound #21

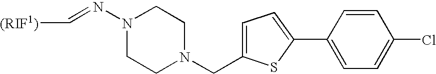

A solution of 3-formyl rifamycin SV (70 mg, 0.10 mmol) and 4-[5-(4-chloro-phenyl)-thiophen-2-ylmethyl]-piperazin-1-ylamine (prepared as in Intermediate Example 22) (90 mg, 0.29 mmol) in THF (1 mL) was stirred at room temperature for 2 h. The resulting mixture was diluted with EtOAc, washed with 5% aqueous NaH₂PO₄ (˜pH 4), dried with Na₂SO₄, and concentrated in vacuo. The resulting residue was purified by MPLC (SiO₂, 1-5% gradient elution, MeOH % in DCM) to yield the title compound.

MS 1015 (M+1)⁺

### EXAMPLE 22

### Compound #22

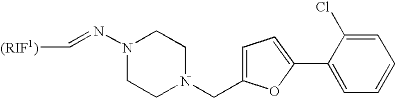

A solution of 3-formyl rifamycin SV (82 mg, 0.11 mmol) and 4-[5-(2-chloro-phenyl)-furan-2-ylmethyl]-piperazin-1-ylamine (prepared as in Intermediate Example 23) (100 mg, 0.34 mmol) in THF (1 mL) was stirred at room temperature for 2 h. The resulting mixture was diluted with EtOAc, washed with 5% aqueous NaH₂PO₄ (˜pH 4), dried with Na₂SO₄, and concentrated in vacuo. The resulting residue was purified by MPLC (SiO₂, 1-5% gradient elution, MeOH % in DCM) to yield the title compound.

MS 999 (M+1)⁺

### EXAMPLE 23

### Compound #23

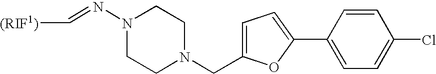

A solution of 3-formyl rifamycin SV (40 mg, 0.05 mmol) and 4-[5-(4-chloro-phenyl)-furan-2-ylmethyl]-piperazin-1-ylamine (prepared as in Intermediate Example 24) (50 mg, 0.17 mmol) in THF (1 mL) was stirred at room temperature for 2 h. The resulting mixture was diluted with EtOAc, washed with 5% aqueous NaH₂PO₄ (˜pH 4), dried with Na₂SO₄, and concentrated in vacuo. The resulting residue was purified by MPLC (SiO₂, 1-5% gradient elution, MeOH % in DCM) to yield the title compound.

MS 999 (M+1)⁺

### EXAMPLE 24

### Compound #24

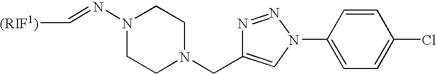

A solution of 3-formyl rifamycin SV (285 mg, 0.39 mmol) and 4-[1-(4-chloro-phenyl)-1H-[1,2,3]triazol-4-ylmethyl]-piperazin-1-ylamine (prepared as in Intermediate Example 25) (150 mg, 0.51 mmol) in THF (2 mL) was stirred at room temperature for 2 h. The resulting mixture was diluted with EtOAc, washed with 5% aqueous NaH₂PO₄ (˜pH 4), dried with Na₂SO₄, and concentrated in vacuo. The resulting residue was purified by MPLC (SiO₂, 1-5% gradient elution, MeOH % in DCM) to yield the title compound.

MS 1000 (M+1)⁺

¹H NMR (400 MHz, CDCl₃): δ 8.26 (s, 1H), 7.94 (s, 1H), 7.70 (d, 2H), 7.52 (d, 2H), 6.56 (dd, 1H), 6.37 (d, 1H), 6.20 (d, 1H), 5.91 (dd, 1H), 5.10 (dd, 1H), 4.94 (d, 1H), 3.84-3.75 (m, 3H), 3.63 (d, 1H), 3.49-3.47 (m, 2H), 3.22-3.00 (m, 4H), 2.79-2.66 (m, 4H),2.42-2.34 (m, 1H), 2.22 (s, 3H), 2.06 (s, 6H), 1.80 (s, 3H), 1.75-1.65 (m, 1H), 1.59-1.49 (m, 1H), 1.41-1.31 (m, 1H), 1.01 (d, 3H), 0.86 (d, 3H), 0.59 (d, 3H), and −0.30 (d, 3H).

### EXAMPLE 25

### Compound #25

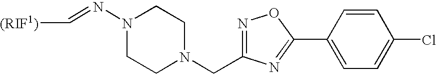

A solution of 3-formyl rifamycin SV (15 mg, 0.02 mmol) and 4-[5-(4-chloro-phenyl)-[1,2,4]oxadiazol-3-ylmethyl]-piperazin-1-ylamine (prepared as in Intermediate Example 26) (18 mg, 0.06 mmol) in THF (1 mL) was stirred at room temperature for 1 h. The resulting mixture was diluted with EtOAc, washed with 5% aqueous NaH₂PO₄ (˜pH 4), dried with Na₂SO₄, and concentrated in vacuo. The resulting residue was purified by MPLC (SiO₂, 1-8% gradient elution, MeOH % in DCM) to yield the title compound.

MS 1001 (M+1)⁺

### EXAMPLE 26

### Compound #26

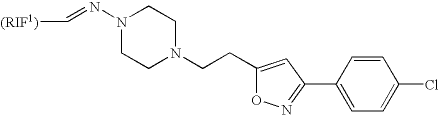

A solution of 3-formyl rifamycin SV (94 mg, 0.13 mmol) and 4-{2-[3-(4-chloro-phenyl)-isoxazol-5-yl]-ethyl}-piperazin-1-ylamine (prepared as in Intermediate Example 27) (100 mg, 0.33 mmol) in THF (1 mL) was stirred at room temperature for 2 h. The resulting mixture was diluted with EtOAc, washed with 5% aqueous NaH₂PO₄ (˜pH 4), dried with Na₂SO₄, and concentrated in vacuo. The resulting residue was purified by MPLC (SiO₂, 1-5% gradient elution, MeOH % in DCM) to yield the title compound.

MS 1014 (M+1)⁺

### EXAMPLE 27

### Compound #27 and Compound #28

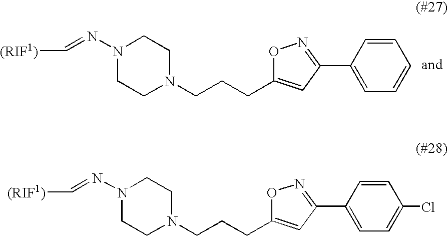

A solution of 3-formyl rifamycin SV (125 mg, 0.17 mmol) and a mixture of 4-[3-(3-phenyl-isoxazol-5-yl)-propyl]-piperazin-1-ylamine and 4-{3-[3-(4-chloro-phenyl)-isoxazol-5-yl]-propyl}-piperazin-1-ylamine (prepared as in Intermediate Example 28) (100 mg, ˜0.33 mmol) in THF (1 mL) was stirred at room temperature for 30 min. The resulting mixture was diluted with EtOAc, washed with 5% aqueous NaH₂PO₄ (˜pH 4), dried with Na₂SO₄, and concentrated in vacuo. The resulting residue was purified by HPLC (C-18, 80-100% gradient elution, MeCN % in water) to yield a mixture of Compound #27 MS 994 (M+1)⁺]; and Compound #28 [MS 1028 (M+1)⁺]

### EXAMPLE 28

### Compound #29

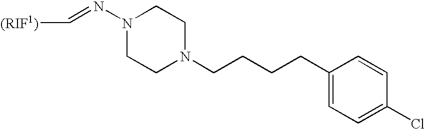

A solution of 3-formyl rifamycin SV (35 mg, 0.05 mmol) and 4-[4-(4-chloro-phenyl)-butyl]-piperazin-1-ylamine (prepared as in Intermediate Example 29) (40 mg, 0.15 mmol) in THF (1 mL) was stirred at room temperature for 2 h. The resulting mixture was diluted with EtOAc, washed with 5% aqueous NaH₂PO₄ (˜pH 4), dried with Na₂SO₄, and concentrated in vacuo. The resulting residue was purified by MPLC (SiO₂, 1-8% gradient elution, MeOH % in DCM) to yield the title compound.

MS 975 (M+1)⁺

### EXAMPLE 29

### Compound #30

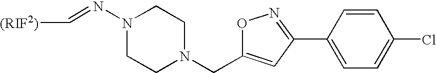

To a solution of Compound #2 (prepared as in Example 2 above) (17 mg, 0.017 mmol) in DCM (0.2 mL) was added activated manganese dioxide (21 mg, 0.23 mmol). The resulting mixture was stirred at room temperature for 1 h, filtered through at bed of CELITE®, the filter bed was rinsed several times with EtOAc, and the combined filtrates were concentrated in vacuo. The resulting residue was purified on MPLC (1-5% gradient elution, MeOH % in DCM) to yield the title compound.

MS 996 (M−1)⁻

### EXAMPLE 30

### Compound #31

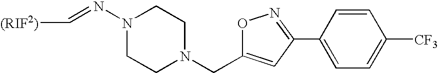

To a solution of Compound #7 (prepared as in Example 7 above) (20 mg, 0.020 mmol) in DCM (0.2 mL) was added activated manganese dioxide (21 mg, 0.23 mmol). The resulting mixture was stirred at room temperature for 1 h, filtered through at bed of CELITE®, the filter bed was rinsed several times with EtOAc, and combined filtrates were concentrated in vacuo. The resulting residue was purified on MPLC (1-5% gradient elution, MeOH % in DCM) to yield the title compound.

MS 1030 (M−1)⁻

### EXAMPLE 32

### Biological Activity

The compounds of the present invention possess antibacterial activity and are therefore useful as antibacterial agents for the treatment of bacterial infections in humans and animals.

Minimum inhibitory concentration (MIC) is an indicator of in vitro antibacterial activity. The MIC is the lowest concentration of test compound that completely inhibits growth of the test organism. The in vitro antimicrobial activity of representative compounds of the present invention was determined by the microdilution broth method following the test method from the National Committee for Clinical Laboratory Standards (NCCLS). This method is described in the NCCLS Document M7-A4, Vol. 17, No. 2, “Methods for Dilution Antimicrobial Susceptibility Test for Bacteria that Grow Aerobically—Fourth Edition”, which is incorporated herein by reference.

In this method two-fold serial dilutions of drug in cation adjusted Mueller-Hinton broth were added to wells in microdilution trays. The test organisms were prepared by adjusting the turbidity of actively growing broth cultures so that the final concentration of test organism after it is added to the wells was approximately 5×10⁴ CFU/well. Following inoculation of the microdilution trays, the trays were incubated at 35° C. for 16-20 hours and then read. The amount of growth in the wells containing the test compound was compared with the amount of growth in the growth-control wells (no test compound) used in each tray.

Representative compounds of the present invention were tested against a variety of pathogenic bacteria with results as listed in Table 3, below.

TABLE 2
In vitro Antibacterial Activity (MIC in μg/mL)

MIC (μg/mL)

| ID No. | A Strain | B Strain | C Strain | D Strain |
|---|---|---|---|---|
| 1 | 0.25 | 0.12 | 0.12 | 0.12 |
| 2 | 1 | 0.5 | 0.5 | 0.5 |
| 3 | 0.5 | 0.5 | 0.5 | 0.5 |
| 4 | 1 | 1 | 0.5 | 1 |
| 5 | 0.25 | 0.25 | 0.12 | 0.12 |
| 6 | 0.5 | 0.5 | 0.25 | 0.25 |
| 7 | 1 | 0.5 | 0.25 | 0.12 |
| 8 | 0.5 | 0.5 | 0.5 | 0.5 |
| 9 | 0.25 | 0.25 | 0.12 | 0.12 |
| 10 | 0.06 | ≦0.03 | ≦0.03 | ≦0.03 |
| 11 | 0.25 | 0.06 | ≦0.03 | ≦0.03 |
| 12 | 0.12 | 0.06 | ≦0.03 | ≦0.03 |
| 13 | 0.06 | ≦0.03 | ≦0.03 | ≦0.03 |
| 14 | 0.06 | ≦0.03 | ≦0.03 | ≦0.03 |
| 15 | 0.06 | ≦0.03 | ≦0.03 | ≦0.03 |
| 16 | 0.12 | 0.06 | 0.06 | ≦0.03 |
| 17 | 0.25 | 0.12 | 0.12 | 0.12 |
| 18 | 1 | 1 | 0.5 | 0.5 |
| 19 | 4 | 1 | 0.5 | 0.5 |
| 20 | 2 | 2 | 1 | 2 |
| 21 | 2 | 2 | 2 | 2 |
| 22 | 0.5 | 0.5 | 0.5 | 0.25 |
| 23 | 0.5 | 0.25 | 0.25 | 0.25 |
| 24 | 0.12 | 0.12 | ≦0.12 | 0.06 |
| 25 | 0.5 | 0.5 | 0.25 | 0.25 |
| 26 | 0.5 | 0.5 | 0.25 | 0.25 |
| 27 | 0.5 | 0.5 | 0.5 | 0.5 |
| 28 | 1 | 1 | 1 | 0.5 |
| 29 | 1 | 0.5 | 1 | 4 |
| 30 | 0.5 | 0.5 | 0.25 | 0.25 |
| 31 | 0.25 | 0.5 | 0.25 | 0.25 |

A: Enterococcus faecium OC 3312 vancomycin-resistant;
B: Staphylococcus aureus ATCC 29213;
C: Methicillin-resistant Staphylococcus aureus OC 3726 COL;
D: Staphylococcus aureus OC4172.
The abbreviation “ND” indicates that the value was not determined.

### EXAMPLE 33

As a specific embodiment of an oral composition, 100 mg of the compound prepared as in Example 10 is formulated with sufficient finely divided lactose to provide a total amount of 580 to 590 mg to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A compound of formula (I) 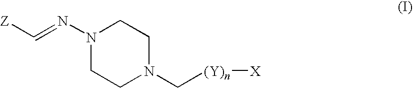
wherein
Z is selected from the group consisting of 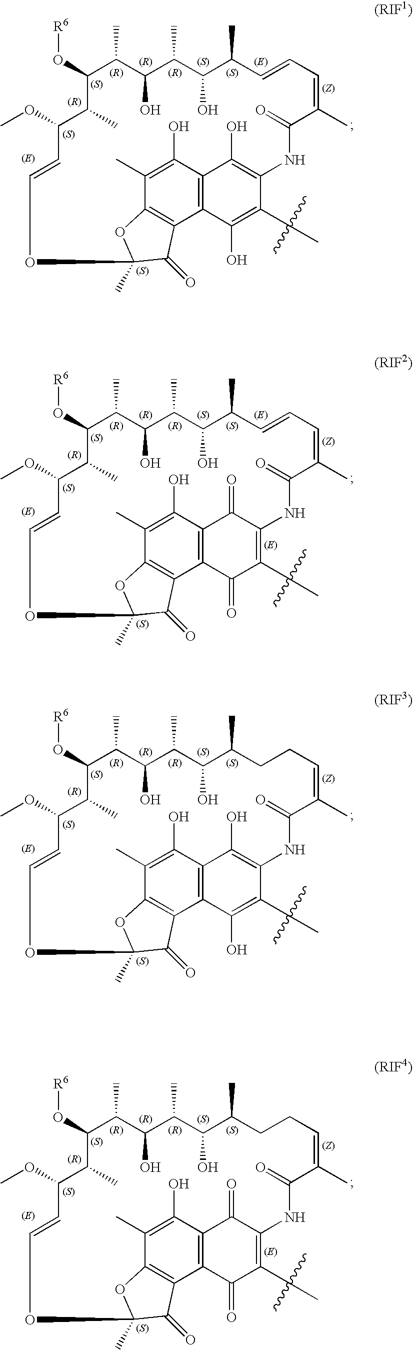 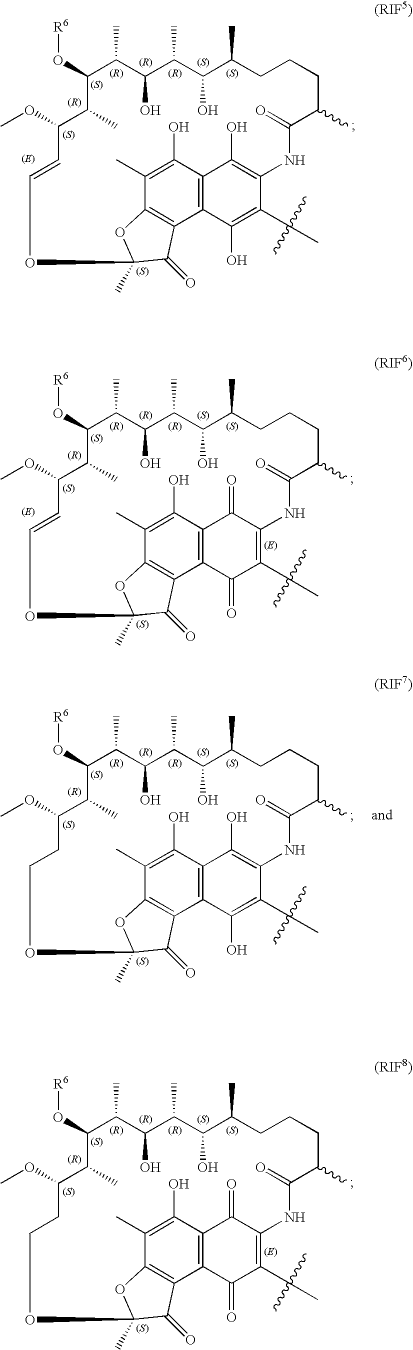
R⁶ is selected from the group consisting of hydrogen and acyl;
n is an integer from 0 to 1;
Y is selected from the group consisting of C₁₋₄alkyl and C₂₋₆alkenyl;
X is selected from the group consisting of R² 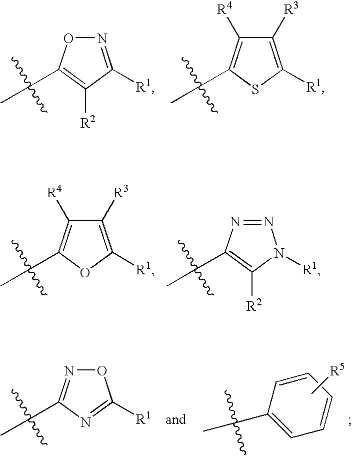
R¹ is selected from the group consisting of C₁₋₄alkyl, —C₁₋₄alkyl-OH, —C₁₋₄alkyl-NR^{A}R^{B}, aryl, heteroaryl, —CO₂H and —CO₂-C₁₋₄alkyl; wherein the aryl is optionally substituted with one to two substituents independently selected from the group consisting of halogen, C₁₋₄alkyl, C₁₋₄alkoxy, halogenated C₁₋₄alkyl, halogenated C₁₋₄alkoxy, —NR^{C}—C(O)—C₁₋₄alkyl and —O—C₁₋₄alkyl-NR^{C}R^{D}; and wherein R^{A}, R^{B}, R^{C} and R^{D} are each independently selected from the group consisting of hydrogen and C₁₋₄alkyl;
R² is selected from the group consisting of hydrogen, —CO₂H and —CO₂—C₁₋₄alkyl;
R³ and R⁴ are each independently selected from the group consisting of hydrogen, halogen, C₁₋₄alkyl, aryl, heteroaryl, —CO₂H and —CO₂-C₁₋₄alkyl;
R⁵ is selected from the group consisting of hydrogen, halogen, C₁₋₄alkyl, aryl, heteroaryl, —CO₂H and —CO₂—C₁₋₄alkyl;or a pharmaceutically acceptable salt, ester or prodrug thereof.

2. A compound as in claim 1, wherein
Z is selected from the group consisting of (RIF¹) and (RIF²);
R⁶ is selected from the group consisting of hydrogen and —C(O)—(C₁₋₄alkyl)
n is an integer from 0 to 1;
Y is selected from the group consisting of C₁₋₄alkyl and C₂₋₄alkenyl;
X is selected from the group consisting of 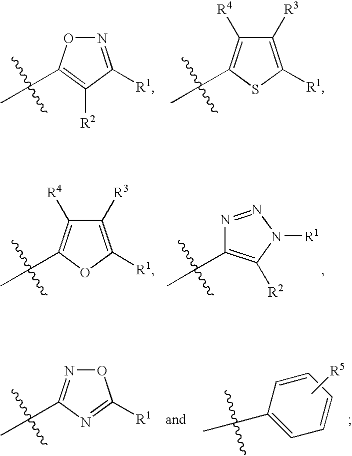
R¹ is selected from the group consisting of C₁₋₄alkyl, —C₁₋₄alkyl-OH, —C₁₋₄alkyl-NR^{A}R^{B}, aryl, heteroaryl, —CO₂H and —CO₂—C₁₋₄alkyl; wherein the aryl is optionally substituted with one to two substituents independently selected from the group consisting of halogen, C₁₋₄alkyl, C₁₋₄alkoxy, halogenated C₁₋₄alkyl, halogenated C₁₋₄alkoxy, —NR^{C}—C(O)—C₁₋₄alkyl and —O—C₁₋₄alkyl-NR^{C}R^{D}; and wherein R^{A}, R^{B}, R^{C} and R^{D} are each independently selected from the group consisting of hydrogen and C₁₋₂alkyl;
R² is selected from the group consisting of hydrogen, —CO₂H and —CO₂—C₁₋₄alkyl;
R³ and R⁴ are each independently selected from the group consisting of hydrogen, C₁₋₄alkyl, phenyl, monocyclic heteroaryl, —CO₂H and —CO₂-C₁₋₄alkyl;
R⁵ is selected from the group consisting of hydrogen, halogen, C₁₋₄alkyl, phenyl, monocyclic heteroaryl, —CO₂H and —CO₂—C₁₋₄alkyl;
or a pharmaceutically acceptable salt, ester or prodrug thereof.

3. A compound as in claim 2, wherein
Z is selected from the group consisting of (RIF¹) and (RIF²);
R⁶ is selected from the group consisting of hydrogen and —C(O)—(C₁₋₄alkyl);
n is an integer from 0 to 1;
Y is selected from the group consisting of C₁₋₄alkyl;
X is selected from the group consisting of 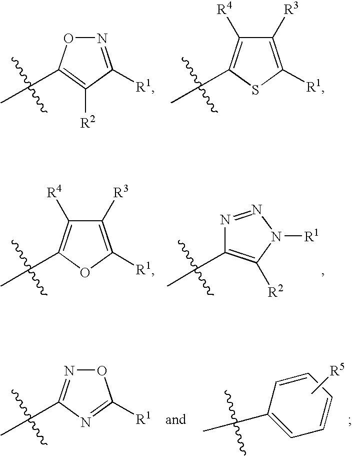
R¹ is selected from the group consisting of C₁₋₄alkyl, —C₁₋₄alkyl-OH, —C₁₋₄alkyl-NR^{A}R^{B}, aryl and heteroaryl; wherein the aryl is optionally substituted with one to two substituents independently selected from the group consisting of halogen, C₁₋₄alkyl, fluorinated C₁₋₄alkyl, fluorinated C₁₋₄alkoxy, —NR^{C}—C(O)—C₁₋₄alkyl and —O—C₁₋₄alkyl-NR^{C}R^{D} ; and wherein R^{A}, R^{B}, R^{C} and R^{D} are each independently selected from the group consisting of hydrogen and C₁₋₂alkyl;
R² is selected from the group consisting of hydrogen, —CO₂H and —CO₂—C₁₋₂alkyl;
R³ and R⁴ are each hydrogen;
R⁵ is selected from the group consisting of halogen;
or a pharmaceutically acceptable salt, ester or prodrug thereof.

4. A compound as in claim 3, wherein
Z is selected from the group consisting of (RIF¹) and (RIF²);
R⁶ is —C(O)—CH₃;
n is an integer from 0 to 1;
Y is selected from the group consisting of —CH₂—, —CH₂CH₂— and —CH₂CH₂CH₂—;
X is selected from the group consisting of 5-(3-phenyl-isoxazolyl), 5-(3-(4-chlorophenyl)-isoxazolyl), 5-(3-(3-chlorophenyl)-isoxazolyl), 5-(3-(2,4-dichlorophenyl)-isoxazolyl), 5-(3-(4-fluorophenyl)-isoxazolyl), 5-(3-(3,4-difluorophenyl)-isoxazolyl), 5-(3-(4-trifluoromethylphenyl)-isoxazolyl), 5-(3-(4-methylphenyl)-isoxazolyl), 5-(3-(4-methoxyphenyl)-isoxazolyl), 5-(3-methyl-isoxazlyl), 5-(3-(hydroxymethyl-isoxazolyl), 5-(3-dimethylaminomethyl-isoxazolyl), 5-(3-(2-pyridyl)-isoxazolyl), 5-(3-(3-pyridyl)-isoxazolyl), 5-(3-(4-pyridyl)-isoxazolyl), 5-(3-(4-methylcarbonyl-amino-phenyl)-isoxazolyl), 5-{3-[4-(3-dimethylaminopropoxy)-phenyl]-isoxazolyl}, 5-(3-(4-chlorophenyl)-4-methoxycarbonyl-isoxazolyl), 5-(3-(4-chlorophenyl)-4-carboxy-isoxazolyl), 2-(5-phenyl-thineyl), 2-(5-(4-chlorophenyl)-isoxazolyl), 2-(5-(2-chlorophenyl)-furyl), 2-(5-(4-chlorophenyl)-furyl), 4-(1-(4-chlorophenyl)-[1,2,3]triazolyl), 3-(5-(4-chlorophenyl)-[1,2,4]oxadiazolyl) and 4-chlorophenyl;
or a pharmaceutically acceptable salt, ester or prodrug thereof.

5. A compound as in claim 1, wherein Z is 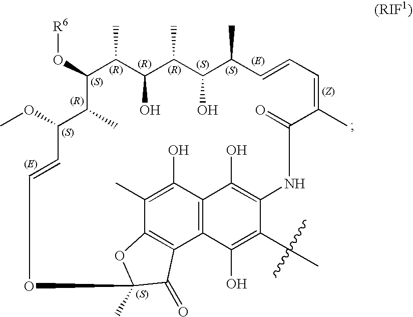
and wherein R⁶ is selected from the group consisting of hydrogen and acyl;

6. A compound as in claim 1, wherein Z is 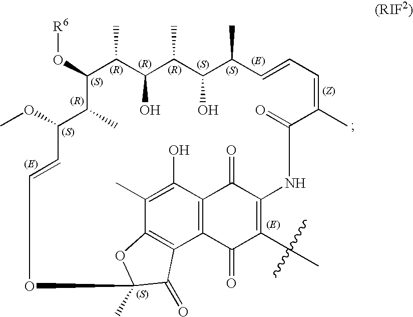
and wherein R⁶ is selected from the group consisting of hydrogen and acyl;

7. A compound selected from the group consisting of 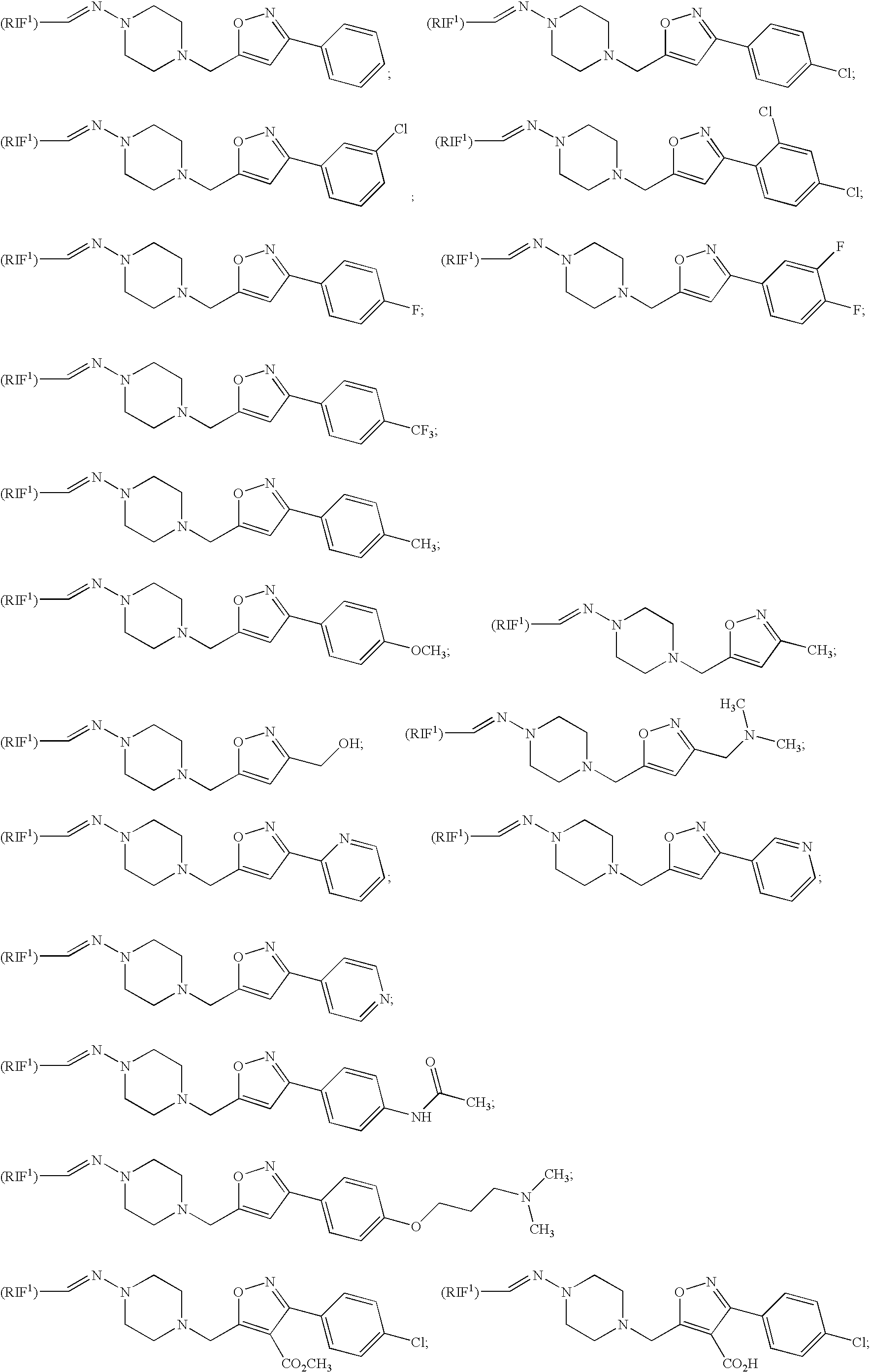 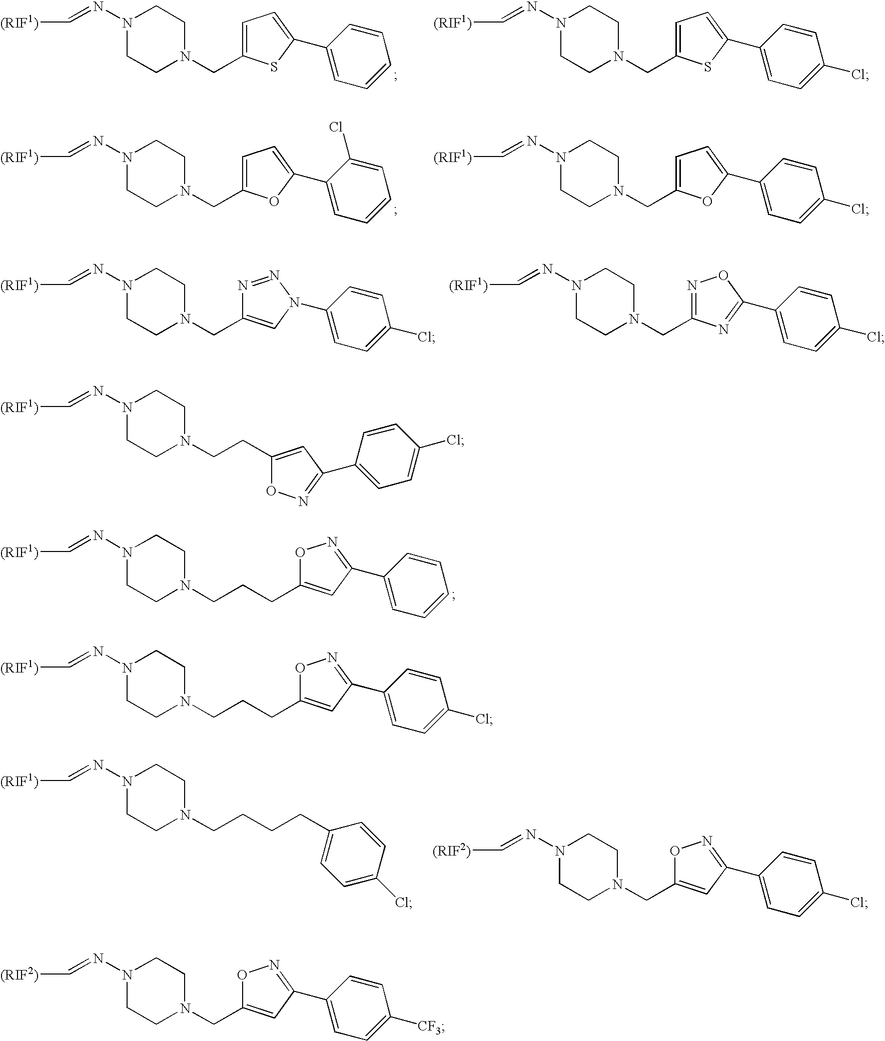
wherein 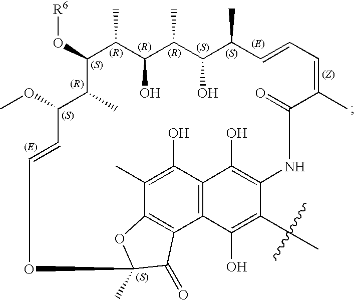
(RIF¹) is 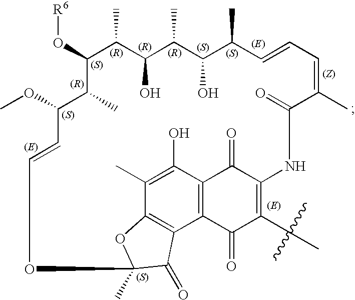
(RIF²) is
R⁶ is —C(O)—CH₃;
and pharmaceutically acceptable salts, esters and prodrug thereof.

8. A compound as in claim 7, selected from the group consisting of 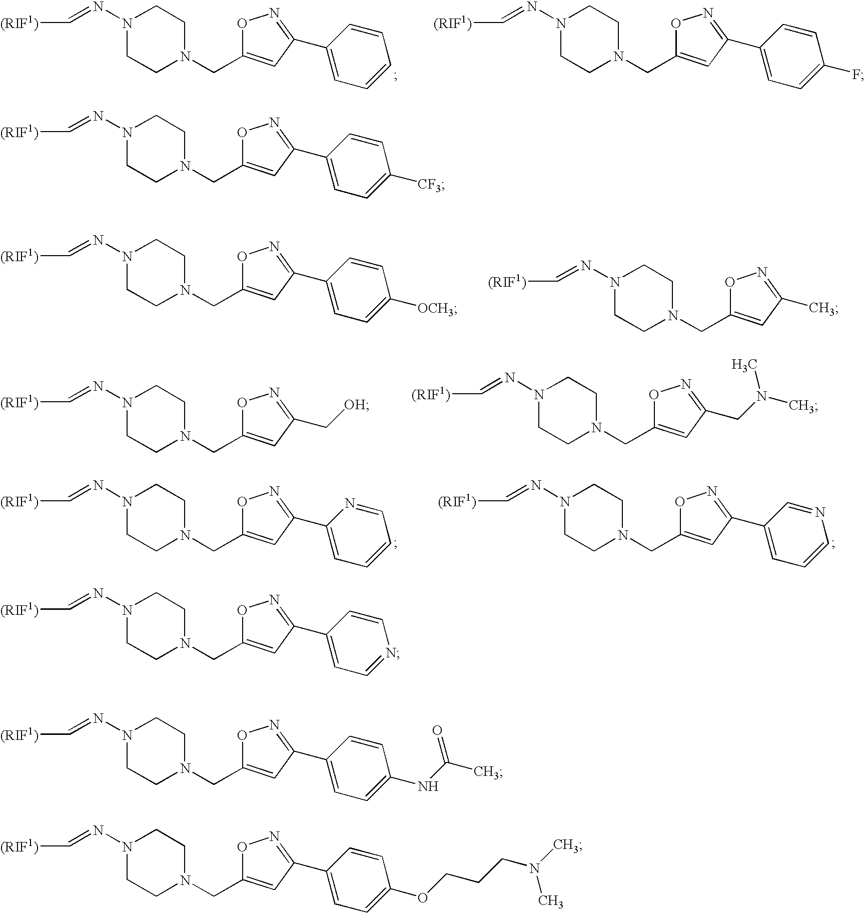
and pharmaceutically acceptable salts, esters and prodrug thereof.

9. A compound as in claim 7, selected from the group consisting of 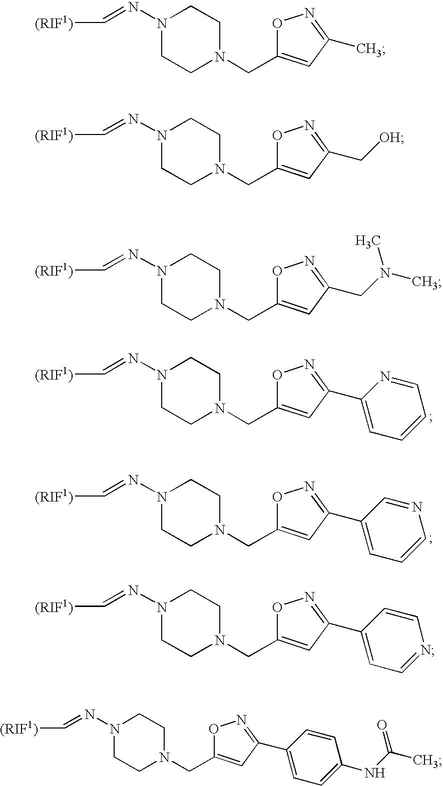
and pharmaceutically acceptable salts, ester and prodrugs thereof.

10. A pharmaceutical composition comprising a pharmaceutically acceptable carrier and a compound of claim 1.

11. A pharmaceutical composition made by mixing a compound of claim 1 and a pharmaceutically acceptable carrier.

12. A process for making a pharmaceutical composition comprising mixing a compound of claim 1 and a pharmaceutically acceptable carrier.

13. A method of treating a subject having a condition caused by or contributed to by bacterial infection, comprising administering to a subject in need thereof a therapeutically effective amount of the compound as in claim 1.

14. A method of preventing a subject from suffering from a condition caused by or contributed to by bacterial infection, comprising administering to a subject in need thereof a prophylactically effective dose of a compound as in claim 1.

15. The use of a compound as in claim 1 for the preparation of a medicament for treating or preventing a condition caused by or contributed to by bacterial infection, in a subject in need thereof.
